(19) United States
(12) Patent Application Publication
(10) Pub. No.: US 2024/0124913 A1
(43) Pub. Date: Apr. 18, 2024

(54) Protein-Based Signal Amplification

(71) Applicant: California Institute of Technology, Pasadena, CA (US)

(72) Inventors: Andrew C. Lu, Pasadena (UA); Michael J. Flynn, Pasadena, CA (US); Lucy S. Chong, Pasadena, CA (US); Ronghui Zhu, Pasadena, CA (US); Michael B. Elowitz, Pasadena, CA (US)

(21) Appl. No.: 18/486,875

(22) Filed: Oct. 13, 2023

Related U.S. Application Data
(60) Provisional application No. 63/416,289, filed on Oct. 14, 2022.

(51) Int. Cl. C12Q 1/37; A61K 38/48; A61P 35/00; C12N 9/22

(52) U.S. Cl. CPC C12Q 1/37; A61K 38/4873; A61P 35/00; C12N 9/22; C12N 2310/20

(57) ABSTRACT

Disclosed herein include methods, compositions, and kits suitable for use in signal amplification. There are provided, in some embodiments, protease-based signal amplification modules. Disclosed herein include amplifier proteins comprising a first part of a first protease domain, a first dimerization domain, a first cut site a protease in a protease active state is capable of cutting, a second dimerization domain, a second cut site a protease in a protease active state is capable of cutting, and a first caging domain. Disclosed herein include companion amplifier proteins comprising a second part of a first protease domain, a third dimerization domain, a third cut site a protease in a protease active state is capable of cutting, a fourth dimerization domain, a fourth cut site a protease in a protease active state is capable of cutting, and a second caging domain.

### RELATED APPLICATIONS

This application claims the benefit under 35 U.S.C. § 119(e) of U.S. Provisional Patent Application Ser. No. 63/416,289, filed Oct. 14, 2022, the content of this related application is incorporated herein by reference in its entirety for all purposes.

### STATEMENT REGARDING FEDERALLY SPONSORED R&D

This invention was made with government support under Grant No. EB030015 awarded by the National Institutes of Health. The government has certain rights in the invention.

### BACKGROUND

### Field

The present disclosure relates generally to the field of synthetic biology.

### Description of the Related Art

Synthetic biology and human cell engineering are poised to revolutionize disease treatment through programmable circuits that can read out cell states and control cellular behaviors. While much pioneering work has been demonstrated with transcriptional circuits, these circuits are limited to operating in the nucleus, thus limiting both the location and type of information accessible. On the other hand, many healthy and diseased cell signaling pathways operate at the protein level in the cytoplasm through protein-protein interactions. Thus, analogous synthetic protein circuits could directly interface with endogenous pathways, unlocking capabilities to sense key cellular pathways, process that information to classify the cellular state, and respond by conditionally triggering cell death or other therapeutic responses. A platform for building synthetic protein circuits based on engineered proteins that interact with one another and with endogenous cellular pathways could provide a powerful platform to perform these functions.

A major obstacle to this paradigm is the inability to reliably sense natural protein signals that are typically present at low concentrations within cells. This problem necessitates complex engineering efforts, often leads to circuits that are noisy, inefficient, or unpredictable, and restricts the repertoire of targets that can be detected. For example, CAR-T cells are currently limited to targeting highly-expressed cell surface proteins (K. Watanabe, et al. (Target antigen density governs the efficacy of anti-CD20-CD28-CD3 chimeric antigen receptor-modified effector CD8+ T cells. J. Immunol. 194, 911-920 (2015))). Intracellularly, synthetic circuits that reliably respond to artificially high levels of an oncogenic signaling pathway can fail to respond when the same pathway is activated at the lower levels present in actual cancer cells. These results suggest the crucial importance of intracellular protein signal amplification. The ability to amplify intrinsically weak signals, and to improve sensitivity more generally, is broadly useful for synthetic biology applications. There is a need for synthetic protein circuit components enabling intracellular protein signal amplification. There is a need for protease-based signal amplification modules.

### SUMMARY

Disclosed herein include synthetic protein circuits. In some embodiments, the synthetic protein circuit comprises: an amplifier protein comprising a first part of a first protease domain, a first dimerization domain, a first cut site a protease in a protease active state is capable of cutting, a second dimerization domain, a second cut site a protease in a protease active state is capable of cutting, and a first caging domain. In some embodiments, the first cut site and/or the second cut site is a cut site a second protease in a second protease active state is capable of cutting. In some embodiments, the synthetic protein circuit comprises: a companion amplifier protein comprising a second part of a first protease domain, a third dimerization domain, a third cut site a protease in a protease active state is capable of cutting, a fourth dimerization domain, a fourth cut site a protease in a protease active state is capable of cutting, and a second caging domain. In some embodiments, the third cut site and/or the fourth cut site is a cut site a second protease in a second protease active state is capable of cutting. In some embodiments, the synthetic protein circuit comprises: one or more input protein(s). In some embodiments, said input protein(s) are configured to detect cell type and/or cell state. In some embodiments, said input protein(s) are capable of constituting a second protease in a second protease active state. In some embodiments, the synthetic protein circuit comprises: one or more output protein(s). In some embodiments, said output protein(s) comprise a cut site the first protease in a first protease active state is capable of cutting, thereby modulating its expression, concentration, localization, stability, and/or activity. In some embodiments, said output protein(s) comprise one or more payload protein(s) and/or effector protein(s). In some embodiments, said output protein(s) are capable of modulating cell type and/or cell state.

In some embodiments, the amplifier protein and the companion amplifier protein separately do not comprise a first protease capable of being in a first protease active state. In some embodiments, the first caging domain is a catalytically inactive version of the second part of a first protease domain. In some embodiments, the first caging domain has between about 50% to about 99% sequence identity to the second part of a first protease domain. In some embodiments, the second caging domain is a catalytically inactive version of the first part of a first protease domain. In some embodiments, the second caging domain has between about 50% to about 99% sequence identity to the first part of a first protease domain. In some embodiments, the first cut site and the second cut site flank the second dimerization domain. In some embodiments, the third cut site and the fourth cut site flank the fourth dimerization domain. In some embodiments, the first dimerization domain is capable of binding the second dimerization domain. In some embodiments, the first dimerization domain is capable of binding the third dimerization domain. In some embodiments, the third dimerization domain is capable of binding the fourth dimerization domain. In some embodiments, the affinity of the first dimerization domain for the second dimerization domain is weaker than the affinity of the first dimerization domain for the third dimerization domain. In some embodiments, the affinity of the third dimerization domain for the fourth dimerization domain is weaker than the affinity of the third dimerization domain for the first dimerization domain.

In some embodiments, intramolecular binding between the first dimerization domain and the second dimerization domain of the first amplifier protein is capable of preventing the first part of the first protease domain from associating with the second part of the first protease domain of the companion amplifier protein to form a first protease in a first protease active state. In some embodiments, intramolecular binding between the third dimerization domain and the fourth dimerization domain of the companion amplifier protein is capable of preventing the second part of the first protease domain from associating with the first part of a first protease domain of the amplifier protein to form a first protease in a first protease active state. In some embodiments, a second protease in a second protease active state is capable of: cleaving the first cut site and/or second cut site of the amplifier protein, thereby forming a cleaved amplifier protein; and/or cleaving the third cut site and/or fourth cut site of the companion amplifier protein, thereby forming a cleaved companion amplifier protein. In some embodiments, a cleaved amplifier protein and a cleaved companion amplifier protein are capable of associating via intermolecular binding of the first dimerization domain and the third dimerization domain to form a first complex, wherein the first complex comprises a first protease capable of being in a first protease active state.

In some embodiments, the first protease and/or the second protease comprises an orthogonal protease (e.g., a viral protease). In some embodiments, the first protease and/or the second protease comprises tobacco etch virus (TEV) protease, tobacco vein mottling virus (TVMV) protease, hepatitis C virus protease (HCVP), derivatives thereof, or any combination thereof. In some embodiments, one or more of the first dimerization domain, second dimerization domain, third dimerization domain, and fourth dimerization domain is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, one or more of the first dimerization domain, second dimerization domain, third dimerization domain, and fourth dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bc1-2 homology domain, a coiled-coil domain, a bZIP domain, portions thereof, variants thereof, or any combination thereof. In some embodiments, one or more of the first dimerization domain, second dimerization domain, third dimerization domain, and fourth dimerization domain is a homodimerization domain or a multimerization domain, optionally a homo- or hetero-dimerizing or multimerizing leucine zipper, a PDZ domains, a SH3 domain, aGBD domain, or any combination thereof. In some embodiments, the first dimerization domain and the third dimerization domain are incapable of associating in the absence of a dimerization ligand. In some embodiments, the first dimerization domain and the third dimerization domain are incapable of associating in the presence of a dimerization ligand. In some embodiments, the affinity of: (i) the first dimerization domain for the second dimerization domain; (ii) the first dimerization domain for the third dimerization domain; and/or (iii) the third dimerization domain for the fourth dimerization domain, is dependent on the local concentration of a dimerization ligand. In some embodiments, the dimerization ligand is a dimeric ligand and/or a small molecule. In some embodiments, the dimerization ligand comprises or is derived from AP1903, AP20187, dimeric FK506, a dimeric FK506-like analog, derivatives thereof, or any combination thereof. In some embodiments, the dimerization ligand enables dose-dependent control of the first protease in the first protease active state.

In some embodiments, the amplifier protein and companion amplifier protein are configured to form an amplification module. In some embodiments, the number of molecules of the first protease in a first protease active state is at least 1.1-fold greater than the number of molecules of the second protease in a second protease active state. In some embodiments, the rate of first protease-mediated cleavage is at least 1.1-fold greater than the rate of second protease-mediated cleavage, In some embodiments, the amplification module thereby achieves signal amplification, optionally in a cell in which a threshold level of input signal is achieved. In some embodiments, configuring the amplifier protein and companion amplifier protein to form an amplification module comprises one or more of: introducing one or more amino acid substitutions into the cut site(s) to increase cleavage efficiency; introducing one or more amino acid substitutions into the first dimerization domain and/or the third dimerization domain to increase affinity for each other; introducing one or more amino acid substitutions into the first dimerization domain and/or the second dimerization domain to decrease affinity for each other; introducing one or more amino acid substitutions into the third dimerization domain and/or the fourth dimerization domain to decrease affinity for each other; introducing one or more amino acid substitutions into the first part of a first protease domain and/or the second part of a first protease domain to increase catalytic activity; and/or increasing the relative levels of the amplifier protein and the companion amplifier protein, optionally said relative levels are capable of being regulated via one or more of expression, localization, and stability, further optionally modulated by an upstream synthetic protein circuit.

In some embodiments, the amplifier protein and companion amplifier protein are configured to form an attenuation module. In some embodiments, the number of molecules of the first protease in a first protease active state is at least 1.1-fold less than the number of molecules of the second protease in a second protease active state. In some embodiments, the rate of first protease-mediated cleavage is at least 1.1-fold less than the rate of second protease-mediated cleavage. In some embodiments, the attenuation module thereby achieves signal attenuation, optionally in a cell in which a threshold level of input signal is achieved. In some embodiments, configuring the amplifier protein and companion amplifier protein to form an attenuation module comprises one or more of: introducing one or more amino acid substitutions into the cut site(s) to decrease cleavage efficiency; introducing one or more amino acid substitutions into the first dimerization domain and/or the third dimerization domain to decrease affinity for each other; introducing one or more amino acid substitutions into the first dimerization domain and/or the second dimerization domain to increase affinity for each other; introducing one or more amino acid substitutions into the third dimerization domain and/or the fourth dimerization domain to increase affinity for each other; introducing one or more amino acid substitutions into the first part of a first protease domain and/or the second part of a first protease domain to decrease catalytic activity; and/or reducing the relative levels of the amplifier protein and the companion amplifier protein, optionally said relative levels are capable of being regulated via one or more of expression, localization, and stability, further optionally modulated by an upstream synthetic protein circuit.

In some embodiments, the presence of the amplification module decreases the level of input signal required for a synthetic protein circuit to generate a given level of output by at least about 1.1-fold as compared to a synthetic protein circuit which does not comprise the amplification module. In some embodiments, the presence of the attenuation module increases the level of input signal required for a synthetic protein circuit to generate a given level of output by at least about 1.1-fold as compared to a synthetic protein circuit which does not comprise the attenuation module. In some embodiments, the presence of the amplification module increases the level of output generated by a given level of input signal by at least about 1.1-fold as compared to a synthetic protein circuit which does not comprise the amplification module. In some embodiments, the presence of the attenuation module decreases the level of output generated by a given level of input signal by at least about 1.1-fold as compared to a synthetic protein circuit which does not comprise the attenuation module.

In some embodiments, the synthetic protein circuit further comprises p chained amplifier proteins and p chained companion amplifier proteins forming p chained amplification modules configured to activate each other in series and thereby achieve signal amplification, wherein p is an integer greater than zero. In some embodiments, the synthetic protein circuit further comprises q chained amplifier proteins and q chained companion amplifier proteins forming q chained attenuation modules configured to activate each other in series at successively attenuated levels and thereby achieve signal attenuation, wherein q is an integer greater than zero. In some embodiments, the synthetic protein circuit further comprises n parallel amplifier proteins and n parallel companion amplifier proteins forming n parallel attenuation modules configured to amplify different input signals independently, wherein n is an integer greater than zero. In some embodiments, the input protein(s) comprise two or more input modules responsive to different input signals, wherein the synthetic protein circuit further comprises m supplemental amplifier proteins and m supplemental companion amplifier proteins forming m supplemental amplifier modules configured to form one or more logic gates selected from the group comprising an OR logic gate, AND logic gate, NOR logic gate, NAND logic gate, IMPLY logic gate, NIMPLY logic gate, XOR logic gate, an XNOR logic gate, wherein m is an integer greater than zero, and wherein the synthetic protein circuit is configured to combinatorially select said different input signals to achieve a desired output.

In some embodiments, the amplifier protein, the companion amplifier protein, the input protein(s), and/or the output protein(s) comprise a linker. In some embodiments, the linker is: is a flexible linker, a rigid linker, or a hybrid linker; is hydrophilic or hydrophobic; is between 1 and 250 amino acids; and/or comprises one or more flexible amino acid residues (e.g., about 1 to about 18 flexible amino acid residues). In some embodiments, the flexible amino acid residues comprise glycine, serine, or a combination thereof. In some embodiments, the linker comprises 3 repeating amino acid subunits or more. In some embodiments, the amplifier protein, the companion amplifier protein, the input protein(s), and/or the output protein(s) is localized to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the amplifier protein, the companion amplifier protein, the input protein(s), and/or the output protein(s) is tethered to an intracellular organelle and/or membranes.

In some embodiments, the amplifier protein, the companion amplifier protein, the input protein(s), and/or the output protein(s) comprise a degradation domain. In some embodiments, the presence of said degradation domain causes said protein to be a destabilized state. In some embodiments, the degradation domain is adjacent to a cut site a protease in an active state is capable of cutting. In some embodiments, the cleavage of said adjacent cut site is capable of inactivating or removing the degradation domain, thereby causing said protein to be in stabilized state. In some embodiments, the cleavage of said adjacent cut site is capable of exposing the degradation domain, and thereby causing said protein to be in destabilized state. In some embodiments, the degradation domain comprises a degron. In some embodiments, the degron comprises an N-degron, a dihydrofolate reductase (DHFR) degron, a FKB protein (FKBP) degron, derivatives thereof, or any combination thereof. In some embodiments, the synthetic protein circuit comprises one or more modulator circuit proteins configured to regulate the expression and/or stability of the amplifier protein and/or the companion amplifier protein in response to the cell type and/or cell state of a cell.

In some embodiments, the synthetic protein circuit is present in a cell. In some embodiments, the cell is a cell of a subject, such as, for example, a subject suffering from a disease or disorder. The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor. In some embodiments, the cell is an in vivo cell, an ex vivo cell, or an in situ cell. In some embodiments, the cell is a eukaryotic cell (e.g. a mammalian cell). In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the first protease in the first protease active state is capable of modulating the cell type and/or cell state of a cell based on the presence and/or amount of a unique cell type and/or a unique cell state in a cell detected via the input protein(s). In some embodiments, the synthetic protein circuit and/or input protein(s) are configured to be responsive to changes in: cell environment. Cell environment can comprise location relative to a target site of a subject and/or changes in the presence and/or absence of cell(s) of interest. Said cell(s) of interest can comprise target-specific antigen(s). In some embodiments, the synthetic protein circuit and/or input protein(s) are configured to be responsive to changes in: one or more signal transduction pathways regulating cell survival, cell growth, cell proliferation, cell adhesion, cell migration, cell metabolism, cell morphology, cell differentiation, apoptosis, or any combination thereof. In some embodiments, the synthetic protein circuit and/or input protein(s) are configured to be responsive to changes in: input(s) of a synthetic receptor system, e.g., Synthetic Notch (SynNotch) receptor, a Modular Extracellular Sensor Architecture (MESA) receptor, a synthekine, Tango, dCas9-synR, a chimeric antigen receptor, or any combination thereof. In some embodiments, the synthetic protein circuit and/or input protein(s) are configured to be responsive to changes in: T cell activity, and T cell activity can comprise one or more of T cell simulation, T cell activation, cytokine secretion, T cell survival, T cell proliferation, CTL activity, T cell degranulation, and T cell differentiation.

In some embodiments, the synthetic protein circuit is: (i) capable of modulating cell states, cell types, and/or cell behaviors, (ii) configured to selectively activate cell death and/or immune recruitment to tumor cells; and/or (iii) is configured to detect the intracellular state of a cell and classify it as tumor or normal based on the levels or activities of relevant molecules or pathways. In some embodiments, a unique cell type and/or a unique cell state comprises a unique gene expression pattern. In some embodiments, the unique cell type and/or unique cell state comprises a unique anatomic location. In some embodiments, the unique cell type and/or the unique cell state comprises anatomically locally unique gene expression. In some embodiments, a unique cell type and/or a unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment. In some embodiments, the senescent cell state is induced by a tumor microenvironment comprises expression of CD57, KRLG1, TIGIT, or any combination thereof. In some embodiments, the unique cell state comprises: (i) a physiological state, optionally a cell cycle state, a differentiation state, a development state a metabolic state, or a combination thereof; and/or (ii) a pathological state, optionally a disease state, a human disease state, a diabetic state, an immune disorder state, a neurodegenerative disorder state, an oncogenic state, or a combination thereof.

In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment. In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of autoimmunity, promotion of fibrosis, promotion of senescence, promotion of autophagy, regulation of cell metabolic reprogramming, regulation of tumor microenvironment signaling, inhibition of cell stemness, survival, and invasion. In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the unique cell state and/or unique cell type is characterized by aberrant signaling of one or more signal transducer(s). In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. In some embodiments, the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof. In some embodiments, the signal transduscer(s) is Wnt/β-catenin, BCR-ABL, P53, AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCy, PLCy, NF-kB, FAK, CREB, αIIIβ3, FcεRI, BAD, p70S6K, STAT1, STAT2, STATS, STATS, STAT6, or any combination thereof. In some embodiments, the disease or disorder is characterized by an aberrant signaling of the first transducer.

In some embodiments, the input protein(s) comprise: a first input protein comprising a first signal transducer binding domain and a first part of a second protease domain, wherein the first signal transducer binding domain is capable of binding a first signal transducer to form a first signal transducer-bound input protein; and a second input protein comprising a second signal transducer binding domain and a second part of the second protease domain, wherein the second signal transducer binding domain is capable of binding a second signal transducer to form a second signal transducer-bound input protein. In some embodiments, the first part of the second protease domain and the second part of the second protease domain have weak association affinity. In some embodiments, the first part of the second protease domain and the second part of the second protease domain are capable of associating with each other to constitute a second protease capable of being in a second protease active state when the first signal transducer and the second signal transducer are in close proximity at an association location. In some embodiments, the first signal transducer binding domain of the first input protein and the second signal transducer binding domain of the second input protein are identical. In some embodiments, the first transducer and the second transducer are identical. In some embodiments, the first signal transducer, the second signal transducer, or both, are capable of being localized at the association location. In some embodiments, the first signal transducer when in a first signal transducer active state, the second signal transducer when in a second signal transducer active state, or both, are capable of being localized at the association location. In some embodiments, the first signal transducer when in a first inactive state, the second signal transducer when in a second inactive state, or both, are capable of being localized at the association location. In some embodiments, the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof.

In some embodiments, the first part of the second protease domain and the second part of the second protease domain have the weak association affinity when the first signal transducer is in a first signal transducer inactive state and/or the second signal transducer inactive state. In some embodiments, the first part of the second protease domain and the second part of the second protease domain are incapable of associating to form the second protease in the second protease active state when the first signal transducer is in a first signal transducer inactive state and/or the second signal transducer is in a second signal transducer inactive state. In some embodiments, a first concentration of the first signal transducer-bound input protein and a second concentration of the second signal transducer-bound input protein at the association location are insufficient for the first part of the second protease domain and the second part of the second protease domain to form an active second protease when the first signal transducer is in a first signal transducer inactive state and/or the second signal transducer is in a second signal transducer inactive state. In some embodiments, the first part of the second protease domain and the second part of the second protease domain are capable of associating with each other to form the second protease in the second protease active state at a threshold first input protein concentration and a threshold second input protein concentration at the association location. In some embodiments, the threshold first input protein concentration and the threshold second input protein concentration at the association location are reached at a threshold signal transducer activation level of the signal transducer. In some embodiments, the level of activation of the effector protein is related to a number of molecules of the effector protein in an effector active state, wherein the first level of activation of the first signal transducer is related to a number of molecules of the first signal transducer in a first transducer active state, and/or wherein the second level of activation of the second signal transducer is related to a number of molecules of the second signal transducer in a second transducer active state.

In some embodiments, the effector protein comprises a cut site the first protease in the first protease active state is capable of cutting. In some embodiments, the effector protein is changed into a effector destabilized state, a effector delocalized state, and/or a effector inactivate state after the first protease in the first protease active state cuts the cut site of the effector protein. In some embodiments, the effector protein comprises a degron, wherein the first protease in the first protease active state is capable of cutting the cut site of the effector protein to expose the degron, and wherein the degron of the effector protein being exposed changes the effector protein to an effector destabilized state. In some embodiments, the effector protein is changed into a effector stabilized state, a effector localized state, and/or a effector activate state after the first protease in the first protease active state cuts the cut site of the effector protein. In some embodiments, the effector protein comprises a degron, wherein the first protease in the first protease active state is capable of cutting the cut site of the effector protein to hide the degron, and wherein the degron of the effector protein being hidden changes the effector protein to an effector stabilized state. In some embodiments, the effector protein in an effector active state is capable of activating an endogenous signal transduction pathway. In some embodiments, the effector protein in an effector active state is capable of inactivating an endogenous signal transduction pathway. In some embodiments, the effector protein comprises Caspase-3, Caspase 7, Caspase-9, Caspase-8, Bax, Bid, Bad, Bak, BCL2L11, p53, PUMA, Diablo/SMAC, S-TRAIL, or any combination thereof.

In some embodiments, the first signal transducer and the second signal transducer belong to the same signal transduction pathway, or a different signal transduction pathway. In some embodiments, the first signal transducer binding domain and/or the second signal transducer binding domain comprises a RAS binding domain (RBD) and/or RAS association domain (RAD). In some embodiments, the first signal transducer binding domain and/or the second signal transducer binding domain comprises a lipid binding domain, optionally a Pleckstrin homology (PH) domain. In some embodiments, the first signal transducer binding domain and/or the second signal transducer binding domain comprises an antibody, an antibody fragment, an scFv, a Fv, a Fab, a (Fab′)₂, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the first signal transducer is capable of binding the first signal transducer binding domain and/or the second signal transducer is capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. In some embodiments, the first signal transducer, the second signal transducer, or both are endogenous proteins. In some embodiments, the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCy, PLCy, NF-kB, FAK, CREB, αIIIβ3, FcεRI, BAD, p70S6K, STAT1, STAT2, STATS, STATS, STAT6, or any combination thereof. In some embodiments, the first signal transducer and/or the second signal transducer are capable of regulating cell survival, cell growth, cell proliferation, cell adhesion, cell migration, cell metabolism, cell morphology, cell differentiation, apoptosis, or any combination thereof. In some embodiments, the first signal transducer, the second signal transducer, or both comprise a RAS protein, optionally the RAS protein is KRAS, NRHAS, HRAS, or any combination thereof. In some embodiments, the first signal transducer, the second signal transducer, or both comprise a lipid, optionally the lipid comprises a phospholipid, further optionally the phospholipid is phosphatidylinositol 3-phosphate. In some embodiments, the effector protein is capable of directly or indirectly inducing cell death in the presence of aberrant signaling. In some embodiments, the effector protein is capable of directly or indirectly inducing cell death when a first level of activation of the first signal transducer is above a first signal transducer activation threshold and/or a second level of activation of the second signal transducer is below a second signal transducer activation threshold.

In some embodiments, a payload protein is capable of modulating the expression, concentration, localization, stability, and/or activity of the one or more endogenous proteins of a cell. In some embodiments, the payload protein is a therapeutic protein or a variant thereof, optionally a therapeutic protein configured to prevent or treat a disease or disorder of a subject, further optionally the subject suffers from a deficiency of said therapeutic protein. In some embodiments, a payload protein comprises: fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof; nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof; a cellular reprogramming factor capable of differentiating a given cell into a desired differentiated state, optionally nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof; an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a secretion tag, optionally the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC; a constitutive signal peptide for protein degradation, optionally PEST; a nuclear localization signal (NLS) or a nuclear export signal (NES); a dosage indicator protein, optionally the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof; a cellular reprogramming factor capable of converting an at least partially differentiated cell to a less differentiated cell, optionally Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof; a programmable nuclease, optionally the synthetic protein circuit senses correction of an aberrant locus by said programmable nuclease and reduces effector protein localization and/or activity, optionally the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions; a CRE recombinase, GCaMP, a cell therapy component, a knock-down gene therapy component, a cell-surface exposed epitope, or any combination thereof; a bispecific T cell engager (BiTE); a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof; a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5; a member of the TNF family of cytokines selected from the group consisting of TNF-alpha, TNF-beta, LT-beta, CD40 ligand, Fas ligand, CD 27 ligand, CD 30 ligand, and 4-1 BBL; a member of the immunoglobulin superfamily of cytokines selected from the group consisting of B7.1 (CD80) and B7.2 (B70); an interferon, optionally the interferon is selected from interferon alpha, interferon beta, or interferon gamma; a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15; an interleukin, optionally the interleukin is selected from IL-10 IL-12, IL-1, IL-6, IL-7, IL-15, IL-2, IL-18 or IL-21; a tumor necrosis factor (TNF), optionally the TNF is selected from TNF-alpha, TNF-beta, TNF-gamma, CD252, CD154, CD178, CD70, CD153, or 4-1BBL; a factor locally down-regulating the activity of endogenous immune cells; is capable of remodeling a tumor microenvironment and/or reducing immunosuppression at a target site of a subject; a chimeric antigen receptor (CAR) or T-cell receptor (TCR); and/or an activity regulator, optionally the activity regulator is capable of reducing T cell activity, optionally the CAR and/or TCR comprises one or more of an antigen binding domain, a transmembrane domain, and an intracellular signaling domain, optionally wherein the intracellular signaling domain comprises a primary signaling domain, a costimulatory domain, or both of a primary signaling domain and a costimulatory domain.

In some embodiments, the activity regulator: comprises a ubiquitin ligase involved in TCR/CAR signal transduction selected from the group comprising c-CBL, CBL-B, ITCH, R F125, R F128, WWP2, or any combination thereof; comprises a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof; is a negative regulatory scaffold/adapter protein selected from the group comprising PAG, LIME, NTAL, LAX31, SIT, GAB2, GRAP, ALX, SLAP, SLAP2, DOK1, DOK2, or any combination thereof; is a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAP1, CRKII, C3G, WAVE2, ARP2/3, ABL, ADAP, RIAM, SKAP55, or any combination thereof; comprises the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof; is targeted to the plasma membrane with a targeting sequence derived from LAT, PAG, LCK, FYN, LAX, CD2, CD3, CD4, CD5, CD7, CD8a, PD1, SRC, LYN, or any combination thereof; and/or reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

In some embodiments, a payload protein is an activity regulator, optionally the activity regulator is capable of reducing T cell activity. In some embodiments, the payload protein comprises a pro-death protein capable of halting cell growth and/or inducing cell death (optionally via apoptosis and/or pyroptosis). In some embodiments, the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments: the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4[(2-chloroethyl)(2-mesyloxyethyl)amino]benzoyl-L-glutamic acid (CMDA), Hydroxy-and amino-aniline mustards, Anthracycline glutamates, and/or Methotrexate α-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-α,γ-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU).

In some embodiments, a payload protein comprises one or more receptors and/or a targeting moiety configured to bind a component of a target site of a subject, optionally the target site is a site of disease or disorder or is proximate to a site of a disease or disorder. In some embodiments, the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the one or more targeting moieties are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C-X-C chemokine receptor type 4, cyclic ADP ribose hydrolase, Cyclin B 1, CYP1B 1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αuβ3, αvβ3, α4β7, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-βI, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

Disclosed herein include nucleic acid compositions. The nucleic acid composition can comprise: one or more polynucleotides encoding a synthetic protein circuit provided herein (or components thereof). In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the one or more polynucleotides are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), (3-kinesin 03-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof. In some embodiments the nucleic acid composition comprises mRNA. In some embodiments, the nucleic acid composition is configured to achieve relative levels of the amplifier protein, the companion amplifier protein, the one or more input protein(s), and/or the one or more output protein(s) desired by a user. In some embodiments, the one or more polynucleotides comprise: one or more first polynucleotides encoding an amplifier protein, one or more second polynucleotides encoding a companion amplifier protein, one or more third polynucleotides encoding one or more input protein(s), and/or one or more fourth polynucleotides encoding one or more output protein(s). In some embodiments, at least two of the one or more polynucleotides are operably linked to a tandem gene expression element.

In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m 1Ψ), and 5-methyl-uridine (m5U). In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors, optionally at least one of the one or more vectors is a viral vector, a plasmid, a transposable element, a naked DNA vector, a lipid nanoparticle (LNP), or any combination thereof, optionally the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and optionally the transposable element is piggybac transposon or sleeping beauty transposon. In some embodiments, the one or more polynucleotides are comprised in the one or more vectors, optionally the one or more polynucleotides are comprised in the same vector and/or different vectors, optionally the one or more polynucleotides are situated on the same nucleic acid and/or different nucleic acids. Disclosed herein include compositions (e.g., pharmaceutical compositions) comprising a nucleic acid composition provided herein.

Disclosed herein include systems for classifying the cell type and/or cell state of a cell, comprising one or more components of the synthetic protein circuits provided herein. Disclosed herein include engineered cells or a population of engineered cells, comprising: a synthetic protein circuit provided herein, one or more components of the synthetic protein circuits provided herein and/or the nucleic acid compositions provided herein. Disclosed herein include methods for classifying the cell type and/or cell state of a cell, comprising: expressing a synthetic protein circuit provided herein or one or more components of the synthetic protein circuits provided herein in the cell. Disclosed herein include methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

In some embodiments, administering comprises: (i) isolating one or more cells from the subject; (ii) contacting said one or more cells with a nucleic acid composition provided herein, thereby generating engineered cells, optionally the contacting comprises transfection; and (iii) administering the one or more engineered cells into a subject after the contacting step. In some embodiments, administering and/or expressing comprises COURIER cellular RNA exporters. In some embodiments, the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, optionally a solid tumor. In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen, optionally the disease associated with expression of a tumor antigen-associated is selected from the group consisting of a proliferative disease, a precancerous condition, a cancer, and a non-cancer related indication associated with expression of the tumor antigen. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

FIG. 1 depicts a non-limiting exemplary schematic illustrating how CHOMP (circuits of hacked orthogonal modular proteases) enables design of composable protein circuit components.

FIG. 2 depicts a non-limiting exemplary schematic of a full circuit wherein (1) TEV protease-based Ras sensor detects Ras clustering on membrane, (2) TVMV protease-based module amplifies signal, and (3) Casp3 cell death executioner induces apoptosis.

FIGS. 3A-3B depicts non-limiting exemplary data related to Ras binding domain (RBD) mediated split-protease reconstitution enabling sensing of Ras in an artificially high ras context but not endogenous levels.

FIG. 4 depicts non-limiting exemplary data related to modeling identifying key parameters that control amplification properties.

FIGS. 5A-5C depicts non-limiting exemplary schematics (FIG. 5A) and data (FIGS. 5B-5C) related to engineered proteases enabling a proteolysis-based amplification module.

FIG. 6 depicts non-limiting exemplary data related to a protease amplifier increasing RBD-circuit sensitivity in a H358 NSCLC KRAS G12C mutant cell line.

FIG. 7 depicts non-limiting exemplary data related to a full circuit enabling sensitive and specific killing of mutant Ras cell lines while sparing wild type cells.

FIGS. 8A-8C depicts non-limiting exemplary schematics and data related to caged split proteases enabling amplification of weak signals. FIG. 8A depicts a non-limiting exemplary schematic showing that a double-caged amplifier protease module is activated by input protease cleavage and removal of caging, catalytically dead halves. FIG. 8B depicts non-limiting exemplary data related to an amplifier module that has low activity in the absence of input signal, and high activity with addition of input signal. FIG. 8C depicts non-limiting exemplary data related to the input output curve of the amplifier showing that, at a given level of input activity, the amplifier increases the signal.

FIGS. 9A-9B depicts non-limiting exemplary schematics and data related to a protease amplifier that enables sensing of endogenous cancer cell line ras activity. FIG. 9A depicts a non-limiting exemplary schematic showing a synthetic amplifier model that can be chained together with the Ras sensor and Casp3 modules to encode sense-amplifier-kill circuit. FIG. 8B depicts non-limiting exemplary data related to a full circuit that induces apoptosis conditionally in mutated Ras cancer cells, but not in wild type Ras HEK293 cells.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include synthetic protein circuits. In some embodiments, the synthetic protein circuit comprises: an amplifier protein comprising a first part of a first protease domain, a first dimerization domain, a first cut site a protease in a protease active state is capable of cutting, a second dimerization domain, a second cut site a protease in a protease active state is capable of cutting, and a first caging domain. In some embodiments, the first cut site and/or the second cut site is a cut site a second protease in a second protease active state is capable of cutting. In some embodiments, the synthetic protein circuit comprises: a companion amplifier protein comprising a second part of a first protease domain, a third dimerization domain, a third cut site a protease in a protease active state is capable of cutting, a fourth dimerization domain, a fourth cut site a protease in a protease active state is capable of cutting, and a second caging domain. In some embodiments, the third cut site and/or the fourth cut site is a cut site a second protease in a second protease active state is capable of cutting. In some embodiments, the synthetic protein circuit comprises: one or more input protein(s). In some embodiments, said input protein(s) are configured to detect cell type and/or cell state. In some embodiments, said input protein(s) are capable of constituting a second protease in a second protease active state. In some embodiments, the synthetic protein circuit comprises: one or more output protein(s). In some embodiments, said output protein(s) comprise a cut site the first protease in a first protease active state is capable of cutting, thereby modulating its expression, concentration, localization, stability, and/or activity. In some embodiments, said output protein(s) comprise one or more payload protein(s) and/or effector protein(s). In some embodiments, said output protein(s) are capable of modulating cell type and/or cell state.

Disclosed herein include nucleic acid compositions. The nucleic acid composition can comprise: one or more polynucleotides encoding a synthetic protein circuit provided herein (or components thereof). Disclosed herein include systems for classifying the cell type and/or cell state of a cell, comprising one or more components of the synthetic protein circuits provided herein. Disclosed herein include engineered cells or a population of engineered cells, comprising: a synthetic protein circuit provided herein, one or more components of the synthetic protein circuits provided herein and/or the nucleic acid compositions provided herein. Disclosed herein include methods for classifying the cell type and/or cell state of a cell, comprising: expressing a synthetic protein circuit provided herein or one or more components of the synthetic protein circuits provided herein in the cell. Disclosed herein include methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

### Protein-Based Signal Amplification

Synthetic biology and human cell engineering are poised to revolutionize disease treatment through programmable circuits that can read out cell states and control cellular behaviors. A major obstacle to this paradigm is the inability to reliably sense natural protein signals that are typically present at low concentrations within cells. The ability to amplify intrinsically weak signals, and to improve sensitivity more generally, is broadly useful for synthetic biology applications.

To address this need, there are provided, in some embodiments, a protease-based amplifier. In some embodiments, the amplifier takes a protease activity as an input, and controls a distinct protease activity as an output. The central module can be a synthetic protease-activatable protease. Each half of a split viral protease (TVMV) can be caged with catalytically inactive complementary protease halves. In its expressed form, both polypeptides can be individually inactive and, due to caging, cannot reconstitute a functional output (TVMV) protease. However, proteolysis catalyzed by the input protease (TEVP) can cleave both caged protease halves between the functional domains and catalytically inactive caging domains. This releases the caging domains, allowing the functional domains to combine to form a functional TVMV protease. Complementary dimerization domains with varying affinities further facilitate this reconstitution in some embodiments.

The protease-activated protease was found to achieve signal amplification (Examples). In the absence of input activity, this “pro-TVMVP” had no measurable protease reporter activity. Upon induction of TEV protease, it became activated to levels comparable to full-length, active TVMVP. At the same level of low input TEVP activity, the pro-TVMVP activated at levels higher than TEVP alone, generating the desired signal amplification. This double caging strategy also works with other protease combinations (e.g. TVMVP-activated TEVP) and can be generalized to a diverse range of proteases with varying specificities to create multiple amplifiers that can operate independently in the same cell.

A key application of the protease amplifier is amplifying intrinsically weak signals from oncogenic signaling pathways. Previously, a protease-based circuit was developed that can detect hyperactivated Ras signaling (X. Gao et al, Programmable Protein Circuits in Living Cells, Science, 2018). In this system, elevated levels of Ras signaling lead to Ras protein nanoclusters on the inner cell membrane. When each half of a split TEV protease is fused to a ras binding domain (RBD), clustering reconstitutes TEV proteases, which can in turn trigger downstream responses. It was found that this RBD-mediated split-protease reconstitution enables sensing of Ras in artificially high Ras context. However, when delivered to cancer cell lines expressing hyperactive mutant ras variants at endogenous levels, the same circuit failed to activate, suggesting the need for signal amplification.

To amplify the intrinsically weak cancer Ras signal, the RBD-Ras activity sensor was coupled with the disclosed engineered TVMVP-based amplifier and, as output, a cell death executioner protein module (Examples). When transfected into cells, the resulting protein circuit selectively kills cancer cell lines with elevated Ras activity (Examples). Further, the circuit can function across multiple mechanisms of Ras activation, and exhibits minimal effects in non-Ras hyperactive cells.

The same type of amplification circuit provided herein can be generalized to sense, amplify, and respond to signals from other oncogenic pathways, cell-type specific signaling cell states, or any user defined input, and to activate arbitrary responses. The results shown in Examples 1 and 2 below demonstrate the utility of the protease-based signal amplification module provided herein and its application to a therapeutic protein circuit.

The amplifier module provided herein enables a wide-variety of circuit functions. Multiple orthogonal protease-amplifiers can be chained together in series to perform multi-stage, high gain signal amplification. Alternatively, multiple amplifiers can be linked together in parallel to amplify different input signals independently. In both cases, the multiple amplification modules can be combined via different types of boolean logic gates (e.g. AND, OR, NOT) to combinatorially select different input signals to achieve the desired output. The amplification module can also be tethered to various intracellular organelles and membranes, or restricted to specific cellular compartments such as cytoplasm and nucleus, acting as an intercompartment signal transmitter. For example, a membrane-localized input signal can activate a cytoplasmic amplifier, transmitting the initial input out to the cytoplasm.

The modular design of the amplifier enables flexibility and tunability. The amplification gain of the amplifier can be tuned down, such that the initial signal becomes attenuated, and the module acts as an attenuator. This can be accomplished, in some embodiments, by weakening the protease catalytic activity, or by changing the dimerization domain affinities, and can be applied in situations where the initial signal is too strong, or where some threshold level of input signal is required. On the other hand, the gain can be increased, in some embodiments, by changing the strengths of the protein zippers, such that the probability of post-cleavage reconstitution of the productive protease halves is increased. The zippers can also be swapped out with small-molecule dependent dimerization domains, such that the amplifier is only active, or is inactive, under drug control, enabling safety switches.

The amplifier module can be linked to any arbitrary downstream output. Because the activation of the amplifier is simply an activated protease, any downstream module that is protease-activatable is capable of accepting the amplified signal in some embodiments. For example, an engineered Caspase 3 module that is activated by the amplifier can be induced to kill by the amplifier. Similarly, other output modalities such as cytokine secretion (RELEASE system) or signaling pathway inhibition are directly compatible with the amplifier.

The protein amplifier can be delivered via multiple methods. The amplifier can be delivered via DNA or RNA transfection with lipid based reagents, either proprietary transfection reagents or lipid nanoparticles. On the other hand, the protein amplifier can also be delivered cell-to-cell with systems such as COURIER (Horns et al., 2023 Cell) or IDLVs, as well as viral methods such as lenti-virus or AAV.

There are provided, in some embodiments, synthetic protein circuits. In some embodiments, the synthetic protein circuit comprises: an amplifier protein comprising a first part of a first protease domain, a first dimerization domain, a first cut site a protease in a protease active state is capable of cutting, a second dimerization domain, a second cut site a protease in a protease active state is capable of cutting, and a first caging domain. In some embodiments, the first cut site and/or the second cut site is a cut site a second protease in a second protease active state is capable of cutting. In some embodiments, the synthetic protein circuit comprises: a companion amplifier protein comprising a second part of a first protease domain, a third dimerization domain, a third cut site a protease in a protease active state is capable of cutting, a fourth dimerization domain, a fourth cut site a protease in a protease active state is capable of cutting, and a second caging domain. In some embodiments, the third cut site and/or the fourth cut site is a cut site a second protease in a second protease active state is capable of cutting. In some embodiments, the synthetic protein circuit comprises: one or more input protein(s). Said input protein(s) can be configured to detect cell type and/or cell state. Said input protein(s) can be capable of constituting a second protease in a second protease active state. In some embodiments, the synthetic protein circuit comprises: one or more output protein(s). Said output protein(s) can comprise a cut site the first protease in a first protease active state is capable of cutting, thereby modulating its expression, concentration, localization, stability, and/or activity. Said output protein(s) can comprise one or more payload protein(s) and/or effector protein(s). Said output protein(s) can be capable of modulating cell type and/or cell state.

In some embodiments, the amplifier protein and the companion amplifier protein separately do not comprise a first protease capable of being in a first protease active state. The first caging domain can be a catalytically inactive version of the second part of a first protease domain. The first caging domain can have between about 50% to about 99% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values) sequence identity to the second part of a first protease domain. The second caging domain can be a catalytically inactive version of the first part of a first protease domain. The second caging domain can have between about 50% to about 99% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values) sequence identity to the first part of a first protease domain. In some embodiments, the first cut site and the second cut site flank the second dimerization domain. In some embodiments, the third cut site and the fourth cut site flank the fourth dimerization domain.

The first dimerization domain can be capable of binding the second dimerization domain. The first dimerization domain can be capable of binding the third dimerization domain The third dimerization domain can be capable of binding the fourth dimerization domain. The affinity of the first dimerization domain for the second dimerization domain can be weaker than the affinity of the first dimerization domain for the third dimerization domain. The affinity of the third dimerization domain for the fourth dimerization domain can be weaker than the affinity of the third dimerization domain for the first dimerization domain. Intramolecular binding between the first dimerization domain and the second dimerization domain of the first amplifier protein can be capable of preventing the first part of the first protease domain from associating with the second part of the first protease domain of the companion amplifier protein to form a first protease in a first protease active state. Intramolecular binding between the third dimerization domain and the fourth dimerization domain of the companion amplifier protein can be capable of preventing the second part of the first protease domain from associating with the first part of a first protease domain of the amplifier protein to form a first protease in a first protease active state. A second protease in a second protease active state can be capable of: cleaving the first cut site and/or second cut site of the amplifier protein, thereby forming a cleaved amplifier protein; and/or cleaving the third cut site and/or fourth cut site of the companion amplifier protein, thereby forming a cleaved companion amplifier protein. A cleaved amplifier protein and a cleaved companion amplifier protein can be capable of associating via intermolecular binding of the first dimerization domain and the third dimerization domain to form a first complex, wherein the first complex comprises a first protease capable of being in a first protease active state. In some embodiments, the first protease and/or the second protease comprises an orthogonal protease (e.g., a viral protease). In some embodiments, the first protease and/or the second protease comprises tobacco etch virus (TEV) protease (e.g., EC 3.4.22.44), tobacco vein mottling virus (TVMV) protease, hepatitis C virus protease (HCVP), derivatives thereof, or any combination thereof.

The systems, methods, compositions, and kits provided herein can, in some embodiments, be employed in concert with the designed heterodimer proteins, monomeric polypeptides capable of forming heterodimer proteins described in PCT Patent Publication No. WO2020093043, entitled, “Orthogonal protein heterodimers,” the content of which is incorporated herein by reference in its entirety. The systems, methods, compositions, and kits provided herein can, in some embodiments, be employed in concert with elements described in Chen et al. (Nature, 2019), Thomas et al. (Journal of the American Chemical Society, 2013), Gradgar and Jerala (Journal of Peptide Science, 2011), Reinke et al (Journal of the American Chemical Society, 2010), and Prehoda et al. (Science, 2000), the contents of which are incorporated herein by reference in their entirety. A pair of dimerization domains capable of binding each other can include a DHD heterodimer a polypeptide and a DHD heterodimer b polypeptide. One or more of the first dimerization domain, second dimerization domain, third dimerization domain, and fourth dimerization domain can be selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_B AAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_B AAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, one or more of the first dimerization domain, second dimerization domain, third dimerization domain, and fourth dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bc1-2 homology domain, a coiled-coil domain, a bZIP domain, portions thereof, variants thereof, or any combination thereof. One or more of the first dimerization domain, second dimerization domain, third dimerization domain, and fourth dimerization domain can be a homodimerization domain or a multimerization domain, optionally a homo- or hetero-dimerizing or multimerizing leucine zipper, a PDZ domains, a SH3 domain, aGBD domain, or any combination thereof.

The first dimerization domain and the third dimerization domain can be incapable of associating in the absence of a dimerization ligand. The first dimerization domain and the third dimerization domain can be incapable of associating in the presence of a dimerization ligand. The affinity of: (i) the first dimerization domain for the second dimerization domain; (ii) the first dimerization domain for the third dimerization domain; and/or (iii) the third dimerization domain for the fourth dimerization domain, can be dependent on the local concentration of a dimerization ligand. The dimerization ligand can be a dimeric ligand and/or a small molecule. The dimerization ligand can comprise or can be derived from AP1903, AP20187, dimeric FK506, a dimeric FK506-like analog, derivatives thereof, or any combination thereof. In some embodiments, the dimerization ligand enables dose-dependent control of the first protease in the first protease active state.

The amplifier protein and companion amplifier protein can be configured to form an amplification module. The number of molecules of the first protease in a first protease active state can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater than the number of molecules of the second protease in a second protease active state. The rate of first protease-mediated cleavage can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater than the rate of second protease-mediated cleavage, In some embodiments, the amplification module thereby achieves signal amplification, optionally in a cell in which a threshold level of input signal is achieved. Configuring the amplifier protein and companion amplifier protein to form an amplification module can comprise one or more of: introducing one or more amino acid substitutions into the cut site(s) to increase cleavage efficiency; introducing one or more amino acid substitutions into the first dimerization domain and/or the third dimerization domain to increase affinity for each other; introducing one or more amino acid substitutions into the first dimerization domain and/or the second dimerization domain to decrease affinity for each other; introducing one or more amino acid substitutions into the third dimerization domain and/or the fourth dimerization domain to decrease affinity for each other; introducing one or more amino acid substitutions into the first part of a first protease domain and/or the second part of a first protease domain to increase catalytic activity; and/or increasing the relative levels of the amplifier protein and the companion amplifier protein, optionally said relative levels are capable of being regulated via one or more of expression, localization, and stability, further optionally modulated by an upstream synthetic protein circuit.

The amplifier protein and companion amplifier protein can be configured to form an attenuation module. The number of molecules of the first protease in a first protease active state can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) less than the number of molecules of the second protease in a second protease active state. The rate of first protease-mediated cleavage can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) less than the rate of second protease-mediated cleavage. In some embodiments, the attenuation module thereby achieves signal attenuation, optionally in a cell in which a threshold level of input signal is achieved. Configuring the amplifier protein and companion amplifier protein to form an attenuation module can comprise one or more of: introducing one or more amino acid substitutions into the cut site(s) to decrease cleavage efficiency; introducing one or more amino acid substitutions into the first dimerization domain and/or the third dimerization domain to decrease affinity for each other; introducing one or more amino acid substitutions into the first dimerization domain and/or the second dimerization domain to increase affinity for each other; introducing one or more amino acid substitutions into the third dimerization domain and/or the fourth dimerization domain to increase affinity for each other; introducing one or more amino acid substitutions into the first part of a first protease domain and/or the second part of a first protease domain to decrease catalytic activity; and/or reducing the relative levels of the amplifier protein and the companion amplifier protein, optionally said relative levels are capable of being regulated via one or more of expression, localization, and stability, further optionally modulated by an upstream synthetic protein circuit. In some embodiments, the presence of the amplification module decreases the level of input signal required for a synthetic protein circuit to generate a given level of output by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a synthetic protein circuit which does not comprise the amplification module. In some embodiments, the presence of the attenuation module increases the level of input signal required for a synthetic protein circuit to generate a given level of output by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a synthetic protein circuit which does not comprise the attenuation module. In some embodiments, the presence of the amplification module increases the level of output generated by a given level of input signal by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a synthetic protein circuit which does not comprise the amplification module. In some embodiments, the presence of the attenuation module decreases the level of output generated by a given level of input signal by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a synthetic protein circuit which does not comprise the attenuation module.

In some embodiments, the synthetic protein circuit further comprises p chained amplifier proteins and p chained companion amplifier proteins forming p chained amplification modules configured to activate each other in series and thereby achieve signal amplification, wherein p is an integer greater than zero. For example, the synthetic protein circuit can comprise 2 chained amplification modules (e.g., a first chained amplification module and a second chained amplification module). Each chained amplification module can comprise a chained amplifier protein and chained companion amplifier protein. An activated amplification module can be capable of activating the first chained amplification module, and the activated first chained amplification module can in turn activate the second chained amplification module. Once activated the second chained amplification module can, in some embodiments, cleave output protein(s). In some embodiments, the synthetic protein circuit further comprises q chained amplifier proteins and q chained companion amplifier proteins forming q chained attenuation modules configured to activate each other in series at successively attenuated levels and thereby achieve signal attenuation, wherein q is an integer greater than zero. In some embodiments, the synthetic protein circuit further comprises n parallel amplifier proteins and n parallel companion amplifier proteins forming n parallel attenuation modules configured to amplify different input signals independently, wherein n is an integer greater than zero. In some embodiments, the input protein(s) comprise two or more input modules responsive to different input signals, wherein the synthetic protein circuit further comprises m supplemental amplifier proteins and m supplemental companion amplifier proteins forming m supplemental amplifier modules configured to form one or more logic gates selected from the group comprising an OR logic gate, AND logic gate, NOR logic gate, NAND logic gate, IMPLY logic gate, NIMPLY logic gate, XOR logic gate, an XNOR logic gate, wherein m is an integer greater than zero, and wherein the synthetic protein circuit is configured to combinatorially select said different input signals to achieve a desired output.

The amplifier protein, the companion amplifier protein, the input protein(s), and/or the output protein(s) can comprise a degradation domain. In some embodiments, the presence of said degradation domain causes said protein to be a destabilized state. The degradation domain can be adjacent to a cut site a protease in an active state is capable of cutting. The cleavage of said adjacent cut site can be capable of inactivating or removing the degradation domain, thereby causing said protein to be in stabilized state. The cleavage of said adjacent cut site can be capable of exposing the degradation domain, and thereby causing said protein to be in destabilized state. The degradation domain can comprise a degron. The degron can comprise an N-degron, a dihydrofolate reductase (DHFR) degron, a FKB protein (FKBP) degron, derivatives thereof, or any combination thereof. The synthetic protein circuit can comprise one or more modulator circuit proteins configured to regulate the expression and/or stability of the amplifier protein and/or the companion amplifier protein in response to the cell type and/or cell state of a cell.

In some embodiments, the amplifier protein, the companion amplifier protein, the input protein(s), and/or the output protein(s) comprise a linker. In some embodiments, the linker is: is a flexible linker, a rigid linker, or a hybrid linker; is hydrophilic or hydrophobic; is between 1 and 250 amino acids; and/or comprises one or more flexible amino acid residues (e.g., about 1 to about 18 flexible amino acid residues). In some embodiments, the flexible amino acid residues comprise glycine, serine, or a combination thereof. In some embodiments, the linker comprises 3 repeating amino acid subunits or more. The amplifier protein, the companion amplifier protein, the input protein(s), and/or the output protein(s) can be localized to one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, Golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof, optionally the amplifier protein, the companion amplifier protein, the input protein(s), and/or the output protein(s) is tethered to an intracellular organelle and/or membranes.

In some embodiments, the synthetic protein circuit is present in a cell. In some embodiments, the cell is a cell of a subject, such as, for example, a subject suffering from a disease or disorder. The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof; a cell derived from a donor. In some embodiments, the cell is an in vivo cell, an ex vivo cell, or an in situ cell. The cell can be a eukaryotic cell (e.g. a mammalian cell). The mammalian cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The first protease in the first protease active state can be capable of modulating the cell type and/or cell state of a cell based on the presence and/or amount of a unique cell type and/or a unique cell state in a cell detected via the input protein(s). The synthetic protein circuit and/or input protein(s) can be configured to be responsive to changes in: cell environment. Cell environment can comprise location relative to a target site of a subject and/or changes in the presence and/or absence of cell(s) of interest. Said cell(s) of interest can comprise target-specific antigen(s). The synthetic protein circuit and/or input protein(s) can be configured to be responsive to changes in: one or more signal transduction pathways regulating cell survival, cell growth, cell proliferation, cell adhesion, cell migration, cell metabolism, cell morphology, cell differentiation, apoptosis, or any combination thereof. The synthetic protein circuit and/or input protein(s) can be configured to be responsive to changes in:input(s) of a synthetic receptor system, e.g., Synthetic Notch (SynNotch) receptor, a Modular Extracellular Sensor Architecture (MESA) receptor, a synthekine, Tango, dCas9-synR, a chimeric antigen receptor, or any combination thereof. The synthetic protein circuit and/or input protein(s) can be configured to be responsive to changes in: T cell activity, and T cell activity can comprise one or more of T cell simulation, T cell activation, cytokine secretion, T cell survival, T cell proliferation, CTL activity, T cell degranulation, and T cell differentiation.

In some embodiments, the synthetic protein circuit is: (i) capable of modulating cell states, cell types, and/or cell behaviors, (ii) configured to selectively activate cell death and/or immune recruitment to tumor cells; and/or (iii) is configured to detect the intracellular state of a cell and classify it as tumor or normal based on the levels or activities of relevant molecules or pathways. A unique cell type and/or a unique cell state can comprise a unique gene expression pattern. The unique cell type and/or unique cell state can comprise a unique anatomic location. The unique cell type and/or the unique cell state can comprise anatomically locally unique gene expression. A unique cell type and/or a unique cell state can be caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. The unique cell state can comprise a senescent cell state induced by a tumor microenvironment. The senescent cell state induced by a tumor microenvironment can comprise expression of CD57, KRLG1, TIGIT, or any combination thereof. In some embodiments, the unique cell state comprises: (i) a physiological state, optionally a cell cycle state, a differentiation state, a development state a metabolic state, or a combination thereof; and/or (ii) a pathological state, optionally a disease state, a human disease state, a diabetic state, an immune disorder state, a neurodegenerative disorder state, an oncogenic state, or a combination thereof.

The unique cell state and/or unique cell type can be characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment. The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. The unique cell state and/or unique cell type can be characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of autoimmunity, promotion of fibrosis, promotion of senescence, promotion of autophagy, regulation of cell metabolic reprogramming, regulation of tumor microenvironment signaling, inhibition of cell stemness, survival, and invasion. In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The unique cell state and/or unique cell type can be characterized by aberrant signaling of one or more signal transducer(s). In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. The aberrant signaling can comprise an aberrant signal of at least one signal transduction pathway regulating cell survival, cell growth, cell proliferation, cell adhesion, cell migration, cell metabolism, cell morphology, cell differentiation, apoptosis, or any combination thereof. The signal transduscer(s) can be Wnt/β-catenin, BCR-ABL, P53, AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCγ, NF-kB, FAK, CREB, αIIIβ3, FcεRI, BAD, p70S6K, STAT1, STAT2, STAT3, STATS, STAT6, or any combination thereof. The disease or disorder can be characterized by an aberrant signaling of the first transducer.

In some embodiments, the input protein(s) comprise: a first input protein comprising a first signal transducer binding domain and a first part of a second protease domain, wherein the first signal transducer binding domain is capable of binding a first signal transducer to form a first signal transducer-bound input protein; and a second input protein comprising a second signal transducer binding domain and a second part of the second protease domain, wherein the second signal transducer binding domain is capable of binding a second signal transducer to form a second signal transducer-bound input protein. In some embodiments, the first part of the second protease domain and the second part of the second protease domain have weak association affinity. The first part of the second protease domain and the second part of the second protease domain can be capable of associating with each other to constitute a second protease capable of being in a second protease active state when the first signal transducer and the second signal transducer are in close proximity at an association location. The first signal transducer binding domain of the first input protein and the second signal transducer binding domain of the second input protein can be identical. The first transducer and the second transducer can be identical. The first signal transducer, the second signal transducer, or both, can be capable of being localized at the association location. The first signal transducer when in a first signal transducer active state, the second signal transducer when in a second signal transducer active state, or both, can be capable of being localized at the association location. The first signal transducer when in a first inactive state, the second signal transducer when in a second inactive state, or both, can be capable of being localized at the association location.

The association location can comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof. In some embodiments, the first part of the second protease domain and the second part of the second protease domain have the weak association affinity when the first signal transducer is in a first signal transducer inactive state and/or the second signal transducer inactive state. The first part of the second protease domain and the second part of the second protease domain can be incapable of associating to form the second protease in the second protease active state when the first signal transducer is in a first signal transducer inactive state and/or the second signal transducer is in a second signal transducer inactive state. A first concentration of the first signal transducer-bound input protein and a second concentration of the second signal transducer-bound input protein at the association location can be insufficient for the first part of the second protease domain and the second part of the second protease domain to form an active second protease when the first signal transducer is in a first signal transducer inactive state and/or the second signal transducer is in a second signal transducer inactive state.

The first part of the second protease domain and the second part of the second protease domain can be capable of associating with each other to form the second protease in the second protease active state at a threshold first input protein concentration and a threshold second input protein concentration at the association location. The threshold first input protein concentration and the threshold second input protein concentration at the association location can be reached at a threshold signal transducer activation level of the signal transducer. In some embodiments, the level of activation of the effector protein is related to a number of molecules of the effector protein in an effector active state, wherein the first level of activation of the first signal transducer is related to a number of molecules of the first signal transducer in a first transducer active state, and/or wherein the second level of activation of the second signal transducer is related to a number of molecules of the second signal transducer in a second transducer active state.

The effector protein can comprise a cut site the first protease in the first protease active state is capable of cutting. The effector protein can be changed into a effector destabilized state, a effector delocalized state, and/or a effector inactivate state after the first protease in the first protease active state cuts the cut site of the effector protein. In some embodiments, the effector protein comprises a degron, wherein the first protease in the first protease active state is capable of cutting the cut site of the effector protein to expose the degron, and wherein the degron of the effector protein being exposed changes the effector protein to an effector destabilized state. The effector protein can be changed into a effector stabilized state, a effector localized state, and/or a effector activate state after the first protease in the first protease active state cuts the cut site of the effector protein. In some embodiments, the effector protein comprises a degron, wherein the first protease in the first protease active state is capable of cutting the cut site of the effector protein to hide the degron, and wherein the degron of the effector protein being hidden changes the effector protein to an effector stabilized state. The effector protein in an effector active state can be capable of activating an endogenous signal transduction pathway. The effector protein in an effector active state can be capable of inactivating an endogenous signal transduction pathway. The effector protein can comprise Caspase-3, Caspase 7, Caspase-9, Caspase-8, Bax, Bid, Bad, Bak, BCL2L11, p53, PUMA, Diablo/SMAC, S-TRAIL, or any combination thereof.

In some embodiments, the first signal transducer and the second signal transducer belong to the same signal transduction pathway, or a different signal transduction pathway. The first signal transducer binding domain and/or the second signal transducer binding domain can comprise a RAS binding domain (RBD) and/or RAS association domain (RAD). The first signal transducer binding domain and/or the second signal transducer binding domain can comprise a lipid binding domain, optionally a Pleckstrin homology (PH) domain. The first signal transducer binding domain and/or the second signal transducer binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. The first signal transducer can be capable of binding the first signal transducer binding domain and/or the second signal transducer can be capable of binding the second signal transducer binding domain following a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof.

The first signal transducer, the second signal transducer, or both can be endogenous proteins. The first signal transducer, the second signal transducer, or both can comprise AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STATS, STAT6, or any combination thereof. The first signal transducer and/or the second signal transducer can be capable of regulating cell survival, cell growth, cell proliferation, cell adhesion, cell migration, cell metabolism, cell morphology, cell differentiation, apoptosis, or any combination thereof. The first signal transducer, the second signal transducer, or both can comprise a RAS protein, optionally the RAS protein is KRAS, NRHAS, HRAS, or any combination thereof. The first signal transducer, the second signal transducer, or both can comprise a lipid, optionally the lipid can comprise a phospholipid, further optionally the phospholipid is phosphatidylinositol 3-phosphate. The effector protein can be capable of directly or indirectly inducing cell death in the presence of aberrant signaling. The effector protein can be capable of directly or indirectly inducing cell death when a first level of activation of the first signal transducer is above a first signal transducer activation threshold and/or a second level of activation of the second signal transducer is below a second signal transducer activation threshold.

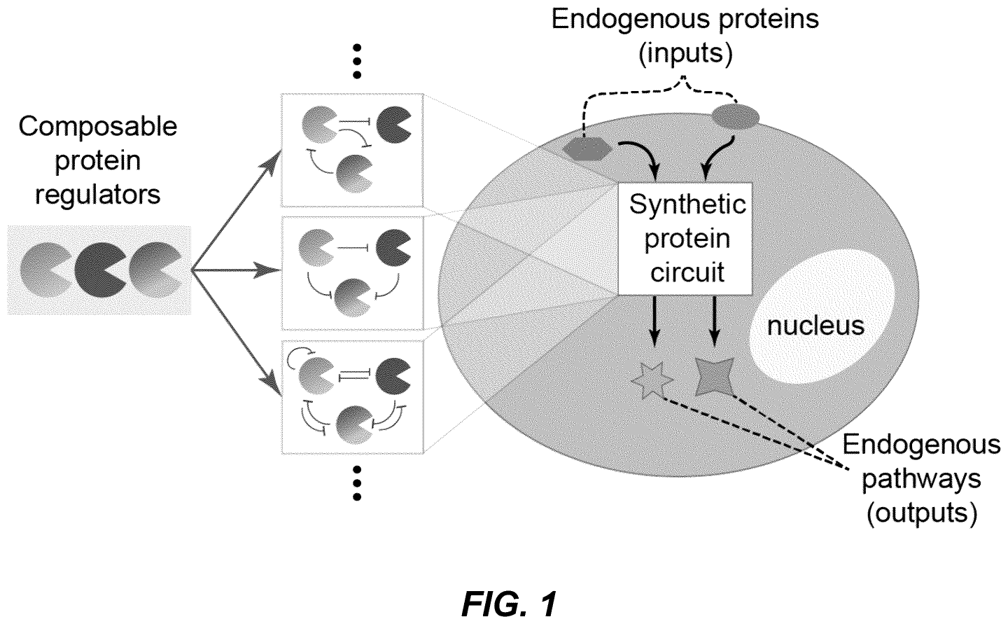

Synthetic biology allows for rational design of circuits that confer new functions in living cells. FIG. 1 depicts a non-limiting exemplary schematic illustrating how CHOMP (circuits of hacked orthogonal modular proteases) enables design of composable protein circuit components. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety. The systems, methods, compositions, and kits provided herein can, in some embodiments, be employed in concert with the systems, methods, compositions, and kits for described in U.S. Patent Publication No. 2023/0076395, entitled, “CELL-TO-CELL DELIVERY OF RNA CIRCUITS,” and in U.S. Patent Publication No. 2023/0071834, entitled, “EXPORTED RNA REPORTERS FOR LIVE-CELL MEASUREMENT,” the contents of which are incorporated herein by reference in their entirety. The systems, methods, compositions, and kits provided herein can, in some embodiments, be employed in concert with the systems, methods, compositions, and kits for described in Chen, Zibo, et al. (“A synthetic protein-level neural network in mammalian cells.” bioRxiv (2022): 2022-07) the content of which is incorporated herein by reference in its entirety. The systems, methods, compositions, and kits provided herein can, in some embodiments, be employed in concert with the systems, methods, compositions, and kits described in PCT Application No. PCT/US23/69663, entitled, “A SYNTHETIC PROTEIN-LEVEL NEURAL NETWORK IN MAMMALIAN CELLS,” filed Jul. 5, 2023, the content of which is incorporated herein by reference in its entirety.

There are provided, in some embodiments, nucleic acid compositions. The nucleic acid composition can comprise: one or more polynucleotides encoding a synthetic protein circuit provided herein (or components thereof). In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the one or more polynucleotides are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof. In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m 1Ψ), and 5-methyl-uridine (m5U).

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors, optionally at least one of the one or more vectors is a viral vector, a plasmid, a transposable element, a naked DNA vector, a lipid nanoparticle (LNP), or any combination thereof, optionally the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and optionally the transposable element is piggybac transposon or sleeping beauty transposon. In some embodiments, the one or more polynucleotides are comprised in the one or more vectors, optionally the one or more polynucleotides are comprised in the same vector and/or different vectors, optionally the one or more polynucleotides are situated on the same nucleic acid and/or different nucleic acids. In some embodiments the nucleic acid composition comprises mRNA. In some embodiments, the nucleic acid composition is configured to achieve relative levels of the amplifier protein, the companion amplifier protein, the one or more input protein(s), and/or the one or more output protein(s) desired by a user. In some embodiments, the one or more polynucleotides comprise: one or more first polynucleotides encoding an amplifier protein, one or more second polynucleotides encoding a companion amplifier protein, one or more third polynucleotides encoding one or more input protein(s), and/or one or more fourth polynucleotides encoding one or more output protein(s). In some embodiments, at least two of the one or more polynucleotides are operably linked to a tandem gene expression element. Disclosed herein include compositions (e.g., pharmaceutical compositions) comprising a nucleic acid composition provided herein.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

Disclosed herein include systems for classifying the cell type and/or cell state of a cell, comprising one or more components of the synthetic protein circuits provided herein. There are provided, in some embodiments, engineered cell(s) or a population of engineered cells, comprising: a synthetic protein circuit provided herein, one or more components of the synthetic protein circuits provided herein and/or the nucleic acid compositions provided herein. There are provided, in some embodiments, methods for classifying the cell type and/or cell state of a cell, comprising: expressing a synthetic protein circuit provided herein or one or more components of the synthetic protein circuits provided herein in the cell.

### Payload Proteins

The output protein(s) can comprise or be a payload protein. A payload protein can comprise an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAGS, PD-1, and/or TIM-3). The one or more payloads can comprise a secretion tag. The secretion tag can be selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. A payload protein can comprise a constitutive signal peptide for protein degradation (e.g., PEST). A payload protein can comprise a nuclear localization signal (NLS) or a nuclear export signal (NES). A payload protein can comprise a dosage indicator protein. The dosage indicator protein can be detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

The payload protein can comprise a synthetic protein circuit component. In some embodiments, the payload comprises a bispecific T cell engager (BiTE). In some embodiments, the orthogonal signal triggers cellular differentiation. The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof. The payload protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the payload protein can diminish immune cell function. The payload protein can be an activity regulator. The activity regulator can be capable of reducing T cell activity. The activity regulator can comprise a ubiquitin ligase involved in TCR/CAR signal transduction selected from the group comprising c-CBL, CBL-B, ITCH, R F125, R F128, WWP2, or any combination thereof. The activity regulator can comprise a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. The activity regulator can be a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. The activity regulator can be a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAP1, CRKII, C3G, WAVE2, ARP2/3, ABL, ADAP, RIAM, SKAP55, or any combination thereof. The activity regulator can comprise the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. The activity regulator can be targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

The payload protein can comprise a cytokine. The cytokine can be selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

The payload protein can comprise a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. The payload protein can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein can comprise a member of the immunoglobulin superfamily of cytokines selected from the group consisting of B7.1 (CD80) and B7.2 (B70). The payload protein can comprise an interferon. The interferon can be selected from interferon alpha, interferon beta, or interferon gamma. The payload protein can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein can comprise a interleukin. The interleukin can be selected from IL-10 IL-12, IL-1, IL-6, IL-7, IL-15, IL-2, IL-18 or IL-21. The payload protein can comprise a tumor necrosis factor (TNF). The TNF can be selected from TNF-alpha, TNF-beta, TNF-gamma, CD252, CD154, CD178, CD70, CD153, or 4-1BBL.

The payload protein can comprise a programmable nuclease. In some embodiments, the synthetic protein circuit senses correction of an aberrant locus by said programmable nuclease and reduces effector protein localization and/or activity. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the synthetic protein circuit comprises a polynucleotide encoding (i) a targeting molecule and/or (ii) a donor nucleic acid. In some embodiments, the targeting molecule is capable of associating with the programmable nuclease. In some embodiments, the targeting molecule comprises single strand DNA or single strand RNA. In some embodiments, the targeting molecule comprises a single guide RNA (sgRNA).

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as—glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload protein is a pro-survival protein (e.g., Bc1-2, Bcl-XL, Mc1-1 and A1). In some embodiments, the payload protein comprises a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload protein is a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload protein is a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload protein is a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1 s, C2b, Bb, D, MASP-1, MASP-2, C4b, C3b, C5a, C3a, C4a, C5b, C6, C7, C8, C9, CR1, CR2, CR3, CR4, C1qR, C1INH, C4bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typ111 repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac 1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein expression in the cell can vary. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

In some embodiments, the payload protein comprises a prodrug-converting enzyme. In some embodiments, the payload protein comprises a pro-death protein capable of halting cell growth and/or inducing cell death (optionally via apoptosis and/or pyroptosis). The pro-death protein can be capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, the pro-death protein is capable of halting cell growth and/or inducing cell death in the presence of a pro-death agent. Any suitable pro-death protein and pro-death agent (e.g., prodrug) is contemplated this disclosure, such as, for example, the suicide gene/prodrug combinations depicted in Table 1. Methods provided herein can comprise administering a prodrug and/or pro-death agent.

TABLE 1
PRO-DEATH PROTEINS AND PRODRUGS

| Pro-death Proteins | Prodrug(s) |
|---|---|
| HSV thymidine kinase (TK) | Ganciclovir (GCV); Ganciclovir elaidic acid ester; |
| | Penciclovir (PCV); Acyclovir (ACV); Valacyclovir (VCV); |
| | (E)-5-(2-bromovinyl)-2′-deoxyuridine (BVDU); Zidovuline |
| | (AZT); 2′-exo-methanocarbathymidine (MCT) |
| Cytosine Deaminase (CD) | 5-fluorocytosine (5-FC) |
| Purine nucleoside | 6-methylpurine deoxyriboside (MEP); fludarabine (FAMP) |
| phosphorylase (PNP) | |
| Cytochrome p450 enzymes | Cyclophosphamide (CPA); Ifosfamide (IFO); 4-ipomeanol |
| (CYP) | (4-IM) |
| Carboxypeptidases (CP) | 4-[(2-chloroethyl)(2-mesyloxyethyl)amino]benzoyl-L- |
| | glutamic acid (CMDA); Hydroxy-and amino-aniline |
| | mustards; Anthracycline glutamates; Methotrexate α- |
| | peptides (MTX-Phe) |
| Caspase-9 | AP1903 |
| Carboxylesterase (CE) | Irinotecan (IRT); Anthracycline acetals |
| Nitroreductase (NTR) | dinitroaziridinylbenzamide CB1954; dinitrobenzamide |
| | mustard SN23862; 4-Nitrobenzyl carbamates; Quinones |
| Horse radish peroxidase | Indole-3-acetic acid (IAA); 5-Fluoroindole-3-acetic acid |
| (HRP) | (FIAA) |
| Guanine Ribosyltransferase | 6-Thioxanthine (6-TX) |
| (XGRTP) | |
| Glycosidase enzymes | HM1826; Anthracycline acetals |
| Methionine-α,γ-lyase (MET) | Selenomethionine (SeMET) |
| Thymidine phosphorylase (TP) | 5′-Deoxy-5-fluorouridine (5'-DFU) |

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both.

Payloads Modulating Signaling Pathways

A cell can be characterized by aberrant signaling of one or more signal transducers. In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. The aberrant signaling can comprise an aberrant signal of at least one signal transduction pathway regulating cell survival, cell growth, cell proliferation, cell adhesion, cell migration, cell metabolism, cell morphology, cell differentiation, apoptosis, or any combination thereof. The disease or disorder can be characterized by an aberrant signaling of the first transducer. The synthetic protein circuits provided herein can be capable of detecting aberrant signaling, an activity of a signal transducer, an activity of a signal transducer activator and/or an activity of a signal transducer repressor. The synthetic protein circuits provided herein can be capable of directly or indirectly inducing cell death in the presence of the aberrant signaling of one or more signal transducer(s). The synthetic protein circuits provided herein can be capable of modulating the degree of signaling in one or more signaling pathways, thereby treating or preventing a disease or disorder. Examples of payload proteins include those associated with a signaling biochemical pathway, e.g., a signaling biochemical pathway-associated gene or polynucleotide (e.g., a signal transducer). Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 2-4. In some embodiments, the methods and compositions disclosed herein prevent or treat one or more of the diseases and disorders listed in Tables 2-4. In some embodiments, the payload(s) and/or synthetic protein circuits provided herein comprises a replacement version of the signal transducer. In some embodiments, the methods and compositions further comprise knockdown of the corresponding endogenous signal transducer. The payload(s) and/or synthetic protein circuits provided herein can comprise the product of a gene listed in listed in Tables 2-4. In some embodiments, the payload(s) and/or synthetic protein circuits provided herein ameliorates a disease or disorder characterized by an aberrant signaling of one or more signaling transducers. In some embodiments, the payload(s) and/or synthetic protein circuits provided herein diminishes the activation level of one or more signal transducers (e.g., signal transducers with aberrant overactive signaling, signal transducers listed in Tables 2-4). In some embodiments, the payload(s) and/or synthetic protein circuits provided herein increases the activation level of one or more signal transducers (e.g., signal transducers with aberrant underactive signaling). In some such embodiments, the payload(s) and/or synthetic protein circuits provided herein can modulate the abundance, location, stability, and/or activity of activators or repressors of said signal transducers.

TABLE 2
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; Notch2; Notch3; |
| | Notch4; AKT; AKT2; AKT3; HIF; HIF1a; HIF3a; Met; HRG; Bcl2; PPAR alpha; |
| | PPAR gamma; WT1 (Wilms Tumor); FGF Receptor Family members (5 |
| | members: 1, 2, 3, 4, 5); CDKN2a; APC; RB (retinoblastoma); MEN1; VHL; |
| | BRCA1; BRCA2; AR (Androgen Receptor); TSG101; IGF; IGF Receptor; Igf1 (4 |
| | variants); Igf2 (3 variants); Igf 1 Receptor; Igf 2 Receptor; Bax; Bcl2; caspases |
| | family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 (Cplx1); Tph1 |
| | Tryptophan hydroxylase; Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; DAOA; DTNBP1; Dao (Dao1) |
| Trinucleotide Repeat | HTT (Huntington's Dx); SBMA/SMAX1/AR (Kennedy's Dx); FXN/X25 |
| Disorders | (Friedrich's Ataxia); ATX3 (Machado-Joseph's Dx); ATXN1 and ATXN2 |
| | (spinocerebellar ataxias); DMPK (myotonic dystrophy); Atrophin-1 and Atn1 |
| | (DRPLA Dx); CBP (Creb-BP—global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related disorders | Prp |
| ALS | SOD1; ALS2; STEX; FUS; TARDBP; VEGF (VEGF-a; VEGF-b; VEGF-c) |
| Drug addiction | Prkce (alcohol); Drd2; Drd4; ABAT (alcohol); GRIA2; Grm5; Grin1; Htr1b; |
| | Grin2a; Drd3; Pdyn; Gria1 (alcohol) |
| Autism | Mecp2; BZRAP1; MDGA2; Sema5A; Neurexin 1; Fragile X (FMR2 (AFF2); |
| | FXR1; FXR2; Mglur5) |
| Alzheimer's Disease | E1; CHIP; UCH; UBB; Tau; LRP; PICALM; Clusterin; PS1; SORL1; CR1; |
| | Vldlr; Uba1; Uba3; CHIP28 (Aqp1, Aquaporin 1); Uchl1; Uchl3; APP |
| Inflammation | IL-10; IL-1 (IL-1a; IL-1b); IL-13; IL-17 (IL-17a (CTLA8); IL17b; IL-17c; IL- |
| | 17d; IL-17f); II-23; Cx3cr1; ptpn22; TNFa; NOD2/CARD15 for IBD; IL-6; IL-12 |
| | (IL-12a; IL-12b); CTLA4; Cx3cl1 |
| Parkinson's Disease | x-Synuclein; DJ-1; LRRK2; Parkin; PINK1 |

TABLE 3
SIGNAL TRANSDUCERS

| | |
|---|---|
| Blood and | Anemia (CDAN1, CDA1, RPS19, DBA, PKLR, PK1, NT5C3, UMPH1, PSN1, RHAG, |
| coagulation | RH50A, NRAMP2, SPTB, ALAS2, ANH1, ASB, ABCB7, ABC7, ASAT); Bare |
| diseases and | lymphocyte syndrome (TAPBP, TPSN, TAP2, ABCB3, PSF2, RING11, MHC2TA, |
| disorders | C2TA, RFX5, RFXAP, RFX5); Bleeding disorders (TBXA2R, P2RX1, P2X1); Factor |
| | H and factor H-like 1 (HF1, CFH, HUS); Factor V and factor VIII (MCFD2); Factor |
| | deficiency (F12, HAF); Factor XIIIA deficiency (F13A1, F13A); Factor XIIIB |
| | VII deficiency (F7); Factor X deficiency (F10); Factor XI deficiency (F11); Factor XII |
| | FAAP90, FLJ34064, FANCB, FANCC, FACC, BRCA2, FANCD1, FANCD2, FANCD, |
| | deficiency (F13B); Fanconi anemia (FANCA, FACA, FA1, FA, FAA, FAAP95, |
| | FACD, FAD, FANCE, FACE, FANCF, XRCC9, FANCG, BRIP1, BACH1, FANCJ, |
| | PHF9, FANCL, FANCM, KIAA1596); Hemophagocytic lymphohistiocytosis disorders |
| | (PRF1, HPLH2, UNC13D, MUNC13-4, HPLH3, HLH3, FHL3); Hemophilia A (F8, |
| | F8C, HEMA); Hemophilia B (F9, HEMB), Hemorrhagic disorders (PI, ATT, F5); |
| | Leukocyde deficiencies and disorders (ITGB2, CD18, LCAMB, LAD, EIF2B1, |
| | EIF2BA, EIF2B2, EIF2B3, EIF2B5, LVWM, CACH, CLE, EIF2B4); Sickle cell anemia |
| | (HBB); Thalassemia (HBA2, HBB, HBD, LCRB, HBA1). |
| Cell | B-cell non-Hodgkin lymphoma (BCL7A, BCL7); Leukemia (TAL1 TCL5, SCL, TAL2, |
| dysregulation | FLT3, NBS1, NBS, ZNFN1A1, IK1, LYF1, HOXD4, HOX4B, BCR, CML, PHL, ALL, |
| and oncology | ARNT, KRAS2, RASK2, GMPS, AF10, ARHGEF12, LARG, KIAA0382, CALM, |
| diseases and | CLTH, CEBPA, CEBP, CHIC2, BTL, FLT3, KIT, PBT, LPP, NPM1, NUP214, D9S46E, |
| disorders | CAN, CAIN, RUNX1, CBFA2, AML1, WHSC1L1, NSD3, FLT3, AF1Q, NPM1, |
| | NUMA1, ZNF145, PLZF, PML, MYL, STAT5B, AF10, CALM, CLTH, ARL11, |
| | ARLTS1, P2RX7, P2X7, BCR, CML, PHL, ALL, GRAF, NF1, VRNF, WSS, NENS, |
| | PTPN11, PTP2C, SHP2, NS1, BCL2, CCND1, PRAD1, BCL1, TCRA, GATA1, GF1, |
| | ERYF1, NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, SDF1); |
| and immune | Autoimmune lymphoproliferative syndrome (TNFRSF6, APT1, FAS, CD95, |
| related | ALPS1A); Combined immunodeficiency, (IL2RG, SCIDX1, SCIDX, IMD4); HIV-1 |
| diseases and | (CCL5, SCYA5, D17S136E, TCP228), HIV susceptibility or infection (IL10, CSIF, |
| disorders | CMKBR2, CCR2, CMKBR5, CCCKR5 (CCR5)); Immunodeficiencies (CD3E, CD3G, |
| | AICDA, AID, HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); Inflammation |
| | (IL-10, IL-1 (IL-1a, IL-1b), IL-13, IL-17 (IL-17a (CTLA8), IL-17b, IL-17c, IL-17d, IL- |
| | 17f), II-23, Cx3cr1, ptpn22, TNFa, NOD2/CARD15 for IBD, IL-6, IL-12 (IL-12a, IL- |
| | 12b), CTLA4, Cx3cl1); Severe combined immunodeficiencies (SCIDs)(JAK3, JAKL, |
| | DCLRE1C, ARTEMIS, SCIDA, RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis (APOA1, APP, AAA, CVAP, AD1, |
| liver, kidney | GSN, FGA, LYZ, TTR, PALB); Cirrhosis (KRT18, KRT8, CIRH1A, NAIC, TEX292, |
| and protein | KIAA1988); Cystic fibrosis (CFTR, ABCC7, CF, MRP7); Glycogen storage diseases |
| diseases and | (SLC2A2, GLUT2, G6PC, G6PT, G6PT1, GAA, LAMP2, LAMPB, AGL, GDE, GBE1, |
| disorders | GYS2, PYGL, PFKM); Hepatic adenoma (TCF1, HNF1A, MODY3), Hepatic failure, |
| | early onset, and neurologic disorder (SCOD1, SCO1), Hepatic lipase deficiency |
| | (LIPC), Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, CASP8, |
| | MCH5); Medullary cystic kidney disease (UMOD, HNFJ, FJHN, MCKD2, |
| | ADMCKD2); Phenylketonuria (PAH, PKU1, QDPR, DHPR, PTS); Polycystic kidney |
| | and hepatic disease (FCYT, PKHD1, ARPKD, PKD1, PKD2, PKD4, PKDTS, PRKCSH, |
| | G19P1, PCLD, SEC63). |
| Muscular/ | Becker muscular dystrophy (DMD, BMD, MYF6), Duchenne Muscular Dystrophy |
| Skeletal | (DMD, BMD); Emery-Dreifuss muscular dystrophy (LMNA, LMN1, EMD2, FPLD, |
| diseases and | CMD1A, HGPS, LGMD1B, LMNA, LMN1, EMD2, FPLD, CMD1A); |
| disorders | Facioscapulohumeral muscular dystrophy (FSHMD1A, FSHD1A); Muscular |
| | dystrophy (FKRP, MDC1C, LGMD2I, LAMA2, LAMM, LARGE, KIAA0609, MDC1D, |
| | FCMD, TTID, MYOT, CAPN3, CANP3, DYSF, LGMD2B, SGCG, LGMD2C, DMDA1, |
| | SCG3, SGCA, ADL, DAG2, LGMD2D, DMDA2, SGCB, LGMD2E, SGCD, SGD, |
| | LGMD2F, CMD1L, TCAP, LGMD2G, CMD1N, TRIM32, HT2A, LGMD2H, FKRP, |
| | MDC1C, LGMD2I, TTN, CMD1G, TMD, LGMD2J, POMT1, CAV3, LGMD1C, |
| | SEPN1, SELN, RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, TIRC7, OC116, |
| | OPTB1); Muscular atrophy (VAPB, VAPC, ALS8, SMN1, SMA1, SMA2, SMA3, |
| | SMA4, BSCL2, SPG17, GARS, SMAD1, CMT2D, HEXB, IGHMBP2, SMUBP2, |
| | CATF1, SMARD1). |
| Neurological | ALS (SOD1, ALS2, STEX, FUS, TARDBP, VEGF (VEGF-a, VEGF-b, VEGF-c); |
| and neuronal | Alzheimer disease (APP, AAA, CVAP, AD1, APOE, AD2, PSEN2, AD4, STM2, |
| diseases and | APBB2, FE65L1, NOS3, PLAU, URK, ACE, DCP1, ACE1, MPO, PACIP1, PAXIP1L, |
| disorders | PTIP, A2M, BLMH, BMH, PSEN1, AD3); Autism (Mecp2, BZRAP1, MDGA2, |
| | Sema5A, Neurexin 1, GLO1, MECP2, RTT, PPMX, MRX16, MRX79, NLGN3, NLGN4, |
| | KIAA1260, AUTSX2); Fragile X Syndrome (FMR2, FXR1, FXR2, mGLUR5); |
| | Huntington's disease and disease like disorders (HD, IT15, PRNP, PRIP, JPH3, JP3, |
| | HDL2, TBP, SCA17); Parkinson disease (NR4A2, NURR1, NOT, TINUR, SNCAIP, |
| | TBP, SCA17, SNCA, NACP, PARK1, PARK4, DJ1, PARK7, LRRK2, PARK8, PINK1, |
| | PARK6, UCHL1, PARK5, SNCA, NACP, PARK1, PARK4, PRKN, PARK2, PDJ, DBH, |
| | NDUFV2); Rett syndrome (MECP2, RTT, PPMX, MRX16, MRX79, CDKL5, STK9, |
| | MECP2, RTT, PPMX, MRX16, MRX79, x-Synuclein, DJ-1); Schizophrenia |
| | (Neuregulin1 (Nrg1), Erb4 (receptor for Neuregulin), Complexin1 (Cplx1), Tph1 |
| | Tryptophan hydroxylase, Tph2, Tryptophan hydroxylase 2, Neurexin 1, GSK3, GSK3a, |
| | GSK3b, 5-HTT (Slc6a4), COMT, DRD (Drd1a), SLC6A3, DAOA, DTNBP1, Dao |
| | (Dao1)); Secretase Related Disorders (APH-1 (alpha and beta), Presenilin (Psen1), |
| | nicastrin, (Ncstn), PEN-2, Nos1, Parp1, Nat1, Nat2); Trinucleotide Repeat Disorders |
| | (HTT (Huntington's Dx), SBMA/SMAX1/AR (Kennedy's Dx), FXN/X25 (Friedrich's |
| | Ataxia), ATX3 (Machado-Joseph's Dx), ATXN1 and ATXN2 (spinocerebellar ataxias), |
| | DMPK (myotonic dystrophy), Atrophin-1 and Atn1 (DRPLA Dx), CBP (Creb-BP—global |
| | instability), VLDLR (Alzheimer's), Atxn7, Atxn10). |
| Ocular | Age-related macular degeneration (Abcr, Ccl2, Cc2, cp (ceruloplasmin), Timp3, |
| diseases and | cathepsinD, Vldlr, Ccr2); Cataract (CRYAA, CRYA1, CRYBB2, CRYB2, PITX3, |
| disorders | BFSP2, CP49, CP47, CRYAA, CRYA1, PAX6, AN2, MGDA, CRYBA1, CRYB1, |
| | CRYGC, CRYG3, CCL, LIM2, MP19, CRYGD, CRYG4, BFSP2, CP49, CP47, HSF4, |
| | CTM, HSF4, CTM, MIP, AQP0, CRYAB, CRYA2, CTPP2, CRYBB1, CRYGD, |
| | CRYG4, CRYBB2, CRYB2, CRYGC, CRYG3, CCL, CRYAA, CRYA1, GJA8, CX50, |
| | CAE1, GJA3, CX46, CZP3, CAE3, CCM1, CAM, KRIT1); Corneal clouding and |
| | dystrophy (APOA1, TGFBI, CSD2, CDGG1, CSD, BIGH3, CDG2, TACSTD2, TROP2, |
| | M1S1, VSX1, RINX, PPCD, PPD, KTCN, COL8A2, FECD, PPCD2, PIP5K3, CFD); |
| | Cornea plana congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, NTG, NPG, |
| | CYP1B1, GLC3A); Leber congenital amaurosis (CRB1, RP12, CRX, CORD2, CRD, |
| | RPGRIP1, LCA6, CORD9, RPE65, RP20, AIPL1, LCA4, GUCY2D, GUC2D, LCA1, |
| | CORD6, RDH12, LCA3); Macular dystrophy (ELOVL4, ADMD, STGD2, STGD3, |
| | RDS, RP7, PRPH2, PRPH, AVMD, AOFMD, VMD2). |

TABLE 4
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; MAPK9; |
| | CDK2; PPP2CA; PIM1; ITGB7; YWHAZ; ILK; TP53; RAF1; IKBKG; RELB; |
| | DYRK1A; CDKN1A; ITGB1; MAP2K2; JAK1; AKT1; JAK2; PIK3R1; |
| | CHUK; PDPK1; PPP2R5C; CTNNB1; MAP2K1; NFKB1; PAK3; ITGB3; |
| | CCND1; GSK3A; FRAP1; SFN; ITGA2; TTK; CSNK1A1; BRAF; GSK3B; |
| | AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid | RAC1; TAF4B; EP300; SMAD2; TRAF6; PCAF; ELK1; MAPK1; SMAD3; |
| Receptor Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; RAC1; RAP1A; |
| Signaling | E1F4E; PRKCZ; NRP1; NTRK2; ARHGEF7; SMO; ROCK2; MAPK1; PGF; |
| | RAC2; PTPN11; GNAS; AKT2; PIK3CA; ERBB2; PRKCI; PTK2; CFL1; |
| | GNAQ; PIK3CB; CXCL12; PIK3C3; WNT11; PRKD1; GNB2L1; ABL1; |
| | MAPK3; ITGA1; KRAS; RHOA; PRKCD; PIK3C2A; ITGB7; GLI2; PXN; |
| | VASP; RAF1; FYN; ITGB1; MAP2K2; PAK4; ADAM17; AKT1; PIK3R1; |
| | GLI1; WNT5A; ADAM10; MAP2K1; PAK3; ITGB3; CDC42; VEGFA; |
| | ITGA2; EPHA8; CRKL; RND1; GSK3B; AKT3; PRKCA |
| Ephrin Receptor | PRKCE; ITGAM; ROCK1; ITGA5; CXCR4; IRAK1; PRKAA2; EIF2AK2; |
| Signaling | RAC1; RAP1A; GRK6; ROCK2; MAPK1; PGF; RAC2; PTPN11; GNAS; |
| | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRKIA; ITGB1; MAP2K2; PAK4, AKT1; JAK2; STAT3; ADAM10; |
| | MAP2K1; PAK3; ITGB3; CDC42; VEGFA; ITGA2; EPHA8; TTK; CSNK1A1; |
| | CRKL; BRAF; PTPN13; ATF4; AKT3; SGK |
| Actin Cytoskeleton | ACTN4; PRKCE; ITGAM; ROCK1; ITGA5; IRAK1; PRKAA2; EIF2AK2; |
| Signaling | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; MAPK9; |
| | CDK2; PIM1; PIK3C2A; ITGB7; PPP1CC; PXN; VIL2; RAF1; GSN; |
| | DYRKIA; ITGB1; MAP2K2; PAK4; PIP5K1A; PIK3R1; MAP2K1; PAK3; |
| | ITGB3; CDC42; APC; ITGA2; TTK; CSNK1A1; CRKL; BRAF; VAV3; SGK |
| Huntington's Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKC1; |
| | HSPA5; REST; GNAQ; PIK3CB; PIK3C3; MAPK8; IGF1R; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; ATF4; |
| | AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; PTPN11; AKT2; IKBKB; |
| Signaling | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; MAP3K7; |
| | MAP2K2; AKT1; PIK3R1; CHUK; MAP2K1; NFKB1; CDC42; GSK3A; |
| | FRAP1; BCL6; BCL10; JUN; GSK3B; ATF4; AKT3; VAV3; RPS6KB1 |
| Leukocyte | ACTN4; CD44; PRKCE; ITGAM; ROCK1; CXCR4; CYBA; RAC1; RAP1A; |
| Extravasation Signaling | PRKCZ; ROCK2; RAC2; PTPN11; MMP14; PIK3CA; PRKCI; PTK2; |
| | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; NOX1; |
| | PXN; VIL2; VASP; ITGB1; MAP2K2; CTNND1; PIK3R1; CTNNB1; CLDN1; |
| | CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; ARHGEF7; |
| | MAPK1; RAC2; CAPNS1; AKT2; CAPN2; PIK3CA; PTK2; PIK3CB; PIK3C3; |
| | MAPK8; CAV1; CAPN1; ABL1; MAPK3; ITGA1; KRAS; RHOA; SRC; |
| | PIK3C2A; ITGB7; PPP1CC; ILK; PXN; VASP; RAF1; FYN; ITGB1; |
| | MAP2K2; PAK4; AKT1; PIK3R1; TNK2; MAP2K1; PAK3; ITGB3; CDC42; |
| | RND3; ITGA2; CRKL; BRAF; GSK3B; AKT3 |
| Acute Phase Response | IRAK1; SOD2; MYD88; TRAF6; ELK1; MAPK1; PTPN11; AKT2; IKBKB; |
| Signaling | PIK3CA; FOS; NFKB2; MAP3K14; PIK3CB; MAPK8; RIPK1; MAPK3; |
| | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; MAP3K7; |
| | MAP2K2; AKT1; JAK2; PIK3R1; CHUK; STAT3; MAP2K1; NFKB1; FRAP1; |
| | CEBPB; JUN; AKT3; IL1R1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| | PIK3CA; CHEK1; TP53INP1; BCL2; PIK3CB; PIK3C3; MAPK8; THBS1; |
| | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAF1; CTNNB1; SIRT1; CCND1; PRKDC; ATM; SFN; |
| | CDKN2A; JUN; SNAI2; GSK3B; BAX; AKT3 |
| Aryl Hydrocarbon | HSPB1; EP300; FASN; TGM2; RXRA; MAPK1; NQO1; NCOR2; SP1; ARNT; |
| Receptor Signaling | CDKN1B; FOS; CHEK1; SMARCA4; NFKB2; MAPK8; ALDH1A1; ATR; |
| | E2F1; MAPK3; NRIP1; CHEK2; RELA; TP73; GSTP1; RB1; SRC; CDK2; |
| | AHR; NFE2L2; NCOA3; TP53; TNF; CDKN1A; NCOA2; APAF1; NFKB1; |
| | CCND1; ATM; ESR1; CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; NCOR2; PIK3CA; ARNT; |
| Signaling | PRKCI; NFKB2; CAMK2A; PIK3CB; PPP2R1A; PIK3C3; MAPK8; PRKD1; |
| | ALDH1A1; MAPK3; NRIP1; KRAS; MAPK13; PRKCD; GSTP1; MAPK9; |
| | NOS2A; ABCB1; AHR; PPP2CA; FTL; NFE2L2; PIK3C2A; PPARGCIA; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK Signaling | PRKCE; IRAK1; PRKAA2; EIF2AK2; RAC1; ELK1; GRK6; MAPK1; |
| | GADD45A; RAC2; PLK1; AKT2; PIK3CA; FADD; CDK8; PIK3CB; PIK3C3; |
| | MAPK8; RIPK1; GNB2L1; IRS1; MAPK3; MAPK10; DAXX; KRAS; |
| | PRKCD; PRKAA1; MAPK9; CDK2; PIM1; PIK3C2A; TRAF2; TP53; LCK; |
| | MAP3K7; DYRK1A; MAP2K2; PIK3R1; MAP2K1; PAK3; CDC42; JUN; |
| | TTK; CSNK1A1; CRKL; BRAF; SGK |
| PPAr/RXR Signaling | PRKAA2; EP300; INS; SMAD2; TRAF6; PPARA; FASN; RXRA; MAPK1; |
| | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; IL1R1; |
| | PRKCA; IL6; HSP90AA1; ADIPOQ |
| NF-KB Signaling | IRAK1; EIF2AK2; EP300; INS; MYD88; PRKCZ: TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4: |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin Signaling | ERBB4; PRKCE; ITGAM; ITGA5: PTEN; PRKCZ; ELK1; MAPK1; PTPN11; |
| | AKT2; EGFR; ERBB2; PRKCI; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPK1; MAP2K1; ITGB3; EREG; FRAP1; |
| | PSEN1; ITGA2; MYC; NRG1; CRKL; AKT3; PRKCA; HSP90AA1; RPS6KB1 |
| Wnt & Beta catenin | CD44; EP300; LRP6; DVL3; CSNK1E; GJA1; SMO; AKT2; PIN1; CDH1; |
| Signaling | BTRC; GNAQ; MARK2; PPP2R1A; WNT11; SRC; DKK1; PPP2CA; SOX6; |
| | SFRP2: ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin Receptor | PTEN; INS; EIF4E; PTPN1; PRKCZ; MAPK1; TSC1; PTPN11; AKT2; CBL; |
| Signaling | PIK3CA; PRKCI; PIK3CB; PIK3C3; MAPK8; IRS1; MAPK3; TSC2; KRAS; |
| | EIF4EBP1; SLC2A4; PIK3C2A; PPP1CC; INSR; RAF1; FYN; MAP2K2; |
| | JAK1; AKT1; JAK2; PIK3R1; PDPK1; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2: MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; RXRA; IKBKB; |
| | PRKCI; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; IL8; |
| | CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKCI; |
| | PTK2; FOS; PIK3CB; PIK3C3; MAPK8; IGF1R; IRS1; MAPK3; IGFBP7; |
| | KRAS; PIK3C2A; YWHAZ; PXN; RAF1; CASP9; MAP2K2; AKT1; PIK3R1; |
| | PDPK1; MAP2K1; IGFBP2; SFN; JUN; CYR61; AKT3; FOXO1; SRF; CTGF; |
| | RPS6KB1 |
| NRF2-mediated | PRKCE; EP300; SOD2; PRKCZ; MAPK1; SQSTM1; NQO1; PIK3CA; PRKCI; |
| Oxidative Stress | FOS; PIK3CB; PIK3C3; MAPK8; PRKD1; MAPK3; KRAS; PRKCD; GSTP1; |
| Response | MAPK9; FTL; NFE2L2; PIK3C2A; MAPK14; RAF1; MAP3K7; CREBBP; |
| | MAP2K2; AKT1; PIK3R1; MAP2K1; PPIB; JUN; KEAP1; GSK3B; ATF4; |
| | PRKCA; EIF2AK3; HSP90AA1 |
| Hepatic | EDN1; IGF1; KDR; FLT1; SMAD2; FGFR1; MET; PGF; SMAD3; EGFR; |
| Fibrosis/Hepatic | FAS; CSF1; NFKB2; BCL2; MYH9; IGF1R; IL6R; RELA; TLR4; PDGFRB; |
| Stellate Cell Activation | TNF; RELB; IL8; PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PPARGC1A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI Signaling | PRKCE; RAC1; PRKCZ; LYN; MAPK1; RAC2; PTPN11; AKT2; PIK3CA; |
| | SYK; PRKCI; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled | PRKCE; RAP1A; RGS16; MAPK1; GNAS; AKT2; IKBKB; PIK3CA; CREB1; |
| Receptor Signaling | GNAQ; NFKB2; CAMK2A; PIK3CB; PIK3C3; MAPK3; KRAS; RELA; SRC; |
| | PIK3C2A; RAF1; IKBKG; RELB; FYN; MAP2K2; AKT1; PIK3R1; CHUK; |
| | PDPK1; STAT3; MAP2K1; NFKB1; BRAF; ATF4; AKT3; PRKCA |
| Inositol Phosphate | PRKCE; IRAK1; PRKAA2; EIF2AK2; PTEN; GRK6; MAPK1; PLK1; AKT2; |
| Metabolism | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; MAPK8; |
| | CAV1; ABL1; MAPK3; KRAS; SRC; PIK3C2A; PDGFRB; RAF1; MAP2K2; |
| | JAK1; JAK2; PIK3R1; PDGFRA; STAT3; SPHK1; MAP2K1; MYC; JUN; |
| | CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer Cell | PRKCE; RAC1; PRKCZ; MAPK1; RAC2; PTPN11; KIR2DL3; AKT2; |
| Signaling | PIK3CA; SYK; PRKCI; PIK3CB; PIK3C3; PRKD1; MAPK3; KRAS; PRKCD; |
| | PTPN6; PIK3C2A; LCK; RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; CDKN1B; BTRC; ATR; ABL1; E2F1; |
| Checkpoint Regulation | HDAC2; HDAC7A; RB1; HDAC11; HDAC9; CDK2; E2F2; HDAC3; TP53; |
| | CDKNIA; CCND1; E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; NFKB2; PIK3CB; |
| Signaling | PIK3C3; MAPK8; MAPK3; KRAS; RELA, PIK3C2A; BTK; LCK; RAF1; |
| | IKBKG; RELB, FYN; MAP2K2; PIK3R1; CHUK; MAP2K1; NFKB1; ITK; |
| | BCL10; JUN; VAV3 |
| Death Receptor | CRADD; HSPB1; BID; BIRC4; TBK1; IKBKB; FADD; FAS; NFKB2; BCL2; |
| Signaling | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3; BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; PIM1; |
| | PIK3C2A; RAF1; MAP2K2; AKT1; JAK2; PIK3R1; STAT3; MAP2K1; |
| | CCND1; AKT3; STAT1 |
| Amyotrophic Lateral | BID; IGF1; RAC1; BIRC4; PGF; CAPNS1; CAPN2; PIK3CA; BCL2; PIK3CB; |
| Sclerosis Signaling | PIK3C3; BCL2L1; CAPN1; PIK3C2A; TP53; CASP9; PIK3R1; RAB5A; |
| | CASP1; APAF1; VEGFA; BIRC2; BAX; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STATA; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; PLK1; AKT2; CDK8; |
| Nicotinamide | MAPK8; MAPK3; PRKCD; PRKAA1; PBEF1; MAPK9; CDK2; PIM1; |
| Metabolism | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPP1CC; MAPK14; |
| | NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; RAF1; |
| | MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKCI; GNAQ; |
| Depression | PPP2R1A; IGF1R; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor | TAF4B; EP300; CARM1; PCAF; MAPK1; NCOR2; SMARCA4; MAPK3; |
| Signaling | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein Ubiquitination | TRAF6; SMURF1; BIRC4; BRCA1; UCHL1; NEDD4; CBL; UBE2I; BTRC; |
| Pathway | HSPA5; USP7; USP10; FBXW7; USP9X; STUB1; USP22; B2M; BIRC2; |
| | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKCI; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor | IRAK1; EIF2AK2; MYD88; TRAF6; PPARA; ELK1; IKBKB; FOS; NFKB2; |
| Signaling | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; IL1R1; SRF; STAT1 |
| Neurotrophin/TRK | NTRK2; MAPK1; PTPN11; PIK3CA; CREB1; FOS; PIK3CB; PIK3C3; |
| Signaling | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKCI; GNAQ; |
| Potentiation | CAMK2A; PRKD1; MAPK3; KRAS; PRKCD; PPP1CC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in | EDN1; PTEN; EP300; NQO1; UBE2I; CREB1; ARNT; HIF1A; SLC2A4; |
| the Cardiovascular | NOS3; TP53; LDHA; AKT1; ATM; VEGFA; JUN; ATF4; VHL; HSP90AA1 |
| System | |
| LPS/IL-1 Mediated | IRAK1; MYD88; TRAF6; PPARA; RXRA; ABCA1, MAPK8; ALDH1A1; |
| Inhibition of RXR | GSTP1; MAPK9; ABCB1; TRAF2; TLR4; TNF; MAP3K7; NR1H2; SREBF1; |
| Function | JUN; IL1R1 |
| LXR/RXR Activation | FASN; RXRA; NCOR2; ABCA1; NFKB2; IRF3; RELA; NOS2A; TLR4; TNF; |
| | RELB; LDLR; NR1H2; NFKB1; SREBF1; IL1R1; CCL2; IL6; MMP9 |
| Amyloid Processing | PRKCE; CSNK1E; MAPK1; CAPNS1; AKT2; CAPN2; CAPN1; MAPK3; |
| | MAPK13; MAPT; MAPK14; AKT1; PSEN1; CSNK1A1; GSK3B; AKT3; APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling | KDR; FLT1; PGF; AKT2; PIK3CA; PIK3CB; PIK3C3; CAV1; PRKCD; NOS3; |
| in the Cardiovascular | PIK3C2A; AKT1; PIK3R1; VEGFA; AKT3; HSP90AA1 |
| System | |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated | RAP1A; MAPK1; GNAS; CREB1; CAMK2A; MAPK3; SRC; RAF1; |
| Signaling | MAP2K2; STAT3; MAP2K1; BRAF; ATF4 |
| Mitochondrial | SOD2; MAPK8; CASP8; MAPK10; MAPK9; CASP9; PARK7; PSEN1; |
| Dysfunction | PARK2; APP; CASP3 |
| Notch Signaling | HES1; JAG1; NUMB; NOTCH4; ADAM17; NOTCH2; PSEN1; NOTCH3; |
| | NOTCH1; DLL4 |
| Endoplasmic Reticulum | HSPA5; MAPK8; XBP1; TRAF2; ATF6; CASP9; ATF4; EIF2AK3; CASP3 |
| Stress Pathway | |
| Pyrimidine Metabolism | NME2; AICDA; RRM2; EIF2AK4; ENTPD1; RRM2B; NT5E; POLD1; NME1 |
| Parkinson's Signaling | UCHL1; MAPK8; MAPK13; MAPK14; CASP9; PARK7; PARK2; CASP3 |
| Cardiac & Beta | GNAS; GNAQ; PPP2R1A; GNB2L1; PPP2CA; PPP1CC; PPP2R5C |
| Adrenergic Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine | PRDX6; PRDX1; TYR |
| and Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; Trpa1; Pomc; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; Wnt3a; Wnt4; |
| Neurology | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4fl or Brn3a); Numb; ReLN |

Chimeric Antigen Receptors and Engineered T Cell Receptors

The payload protein(s) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracelluar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GaLNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Pharmaceutically Acceptable Compositions and Methods

Disclosed herein include pharmaceutical compositions. In some embodiments, the pharmaceutical composition comprises: a composition provided herein (e.g., a nucleic acid composition, a population of engineered cells), wherein the pharmaceutical composition further comprises one or more pharmaceutically acceptable carriers, diluents and/or excipients. There are provided, in some embodiments, methods for detecting a disease or disorder in a subject, comprising: expressing a synthetic protein circuit provided herein or one or more components of the synthetic protein circuits provided herein in a cell of the subject. Disclosed herein include methods for treating or preventing a disease or disorder in a subject in need thereof, comprising: expressing a synthetic protein circuit provided herein or one or more components of the synthetic protein circuits provided herein in a cell of a subject in need thereof. Disclosed herein include methods for treating or preventing a disease or disorder in a subject in need thereof, comprising: administering to the subject an effective amount of a nucleic acid composition provided herein or engineered cells provided herein, thereby treating or preventing the disease or disorder in the subject.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., engineered cells, nucleic acid composition) which can be combined with a carrier material to produce a single dosage form will vary depending upon the host being treated, the particular mode of administration. The amount of active ingredient, which can be combined with a carrier material to produce a single dosage form will generally be that amount of the nucleic acid composition and/or engineered cells which produces a therapeutic effect. Generally, out of one hundred percent, this amount will range from about 1% to about 99% of active ingredient, preferably from about 5% to about 70%, most preferably from about 10% to about 30%.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the engineered cells disclosed herein, thereby treating or preventing the disease or disorder in the subject. In some embodiments, administering comprises: (i) isolating one or more cells from the subject; (ii) contacting (e.g., transfecting) said one or more cells with a nucleic acid composition disclosed herein, thereby generating engineered cells; and (iii) administering the one or more engineered cells into a subject after the contacting step. The method can comprise: administering to the subject an effective amount of a pro-death agent, or any combination thereof. The engineered sends can be configured to travel to and/or accumulate at a target site of a subject. In some embodiments, nucleic acid composition(s) are administered to a subject to generate engineered cells in vivo. Alternatively, in some embodiments, engineered cells are generated (e.g., by incorporating the nucleic acid composition(s) provided herein) outside the body of the subject and are subsequently administrated to the subject.

The disclosed engineered cells described herein may be included in a composition for therapy. In some embodiments, the composition comprises a population of disclosed engineered cells. The composition may include a pharmaceutical composition and further include a pharmaceutically acceptable carrier. A therapeutically effective amount of the pharmaceutical composition comprising the disclosed engineered cells may be administered. The cells provided herein may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating an engineered population of cells. The disclosed engineered cells can be administered to a mammalian recipient to provide a therapeutic benefit. The mammalian recipient may be a human and the disclosed engineered cells can be autologous with respect to the recipient. Alternatively, the disclosed engineered cells can be allogeneic, syngeneic or xenogeneic with respect to the recipient.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. Administering and/or expressing can comprise COURIER cellular RNA exporters. The disclosed engineered cells can be administered at a therapeutically effective amount. For example, a therapeutically effective amount of the disclosed engineered cells can be at least about 10² cells, at least about 10³ cells, at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the disclosed engineered cells is about 10⁴ cells, about 10⁵ cells, about 10⁶ cells, about 10⁷ cells, or about 10⁸ cells. In one particular embodiment, the therapeutically effective amount of the disclosed engineered cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, a solid tumor, or any combination thereof. The disease or disorder can be an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen. The disease associated with expression of a tumor antigen-associated can be selected from the group consisting of a proliferative disease, a precancerous condition, a cancer, and a non-cancer related indication associated with expression of the tumor antigen. The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

### EXAMPLES

Some aspects of the embodiments discussed above are disclosed in further detail in the following examples, which are not in any way intended to limit the scope of the present disclosure.

### Example 1

### Targeting Cancer Cells with Synthetic Protein Circuits

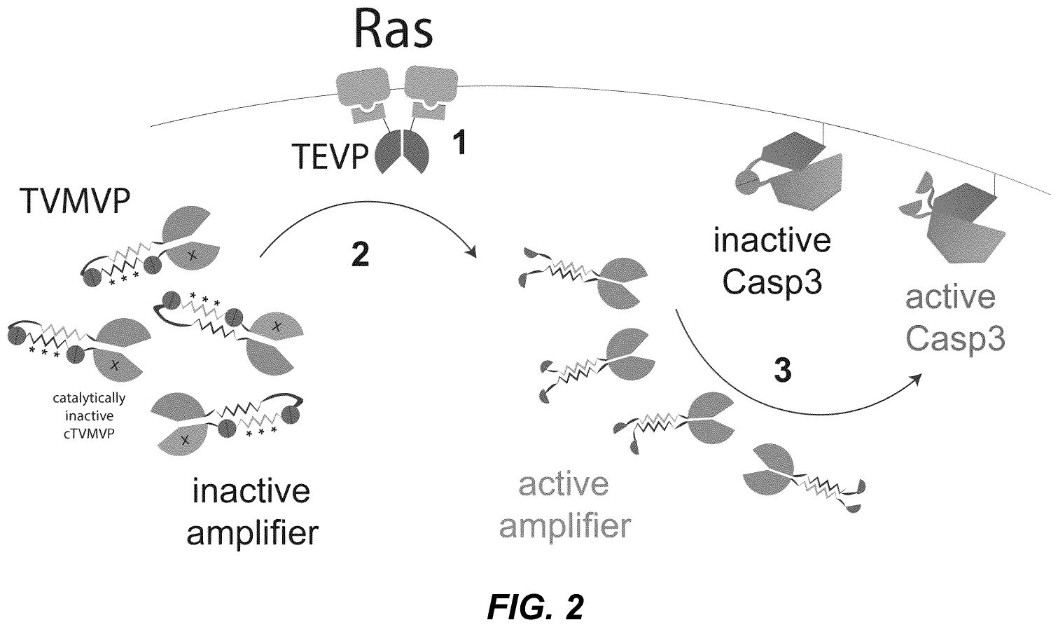

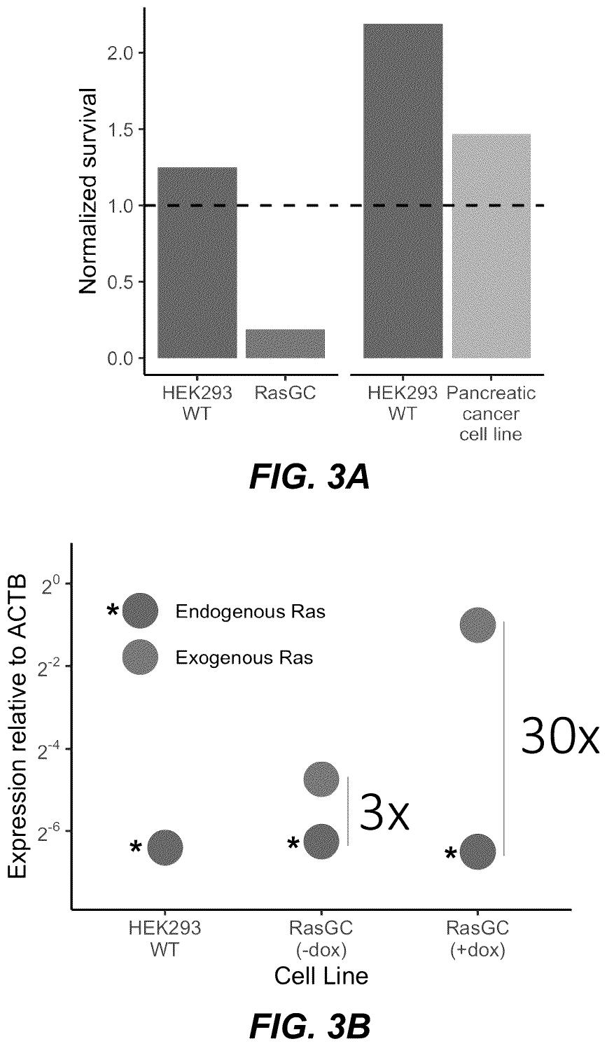

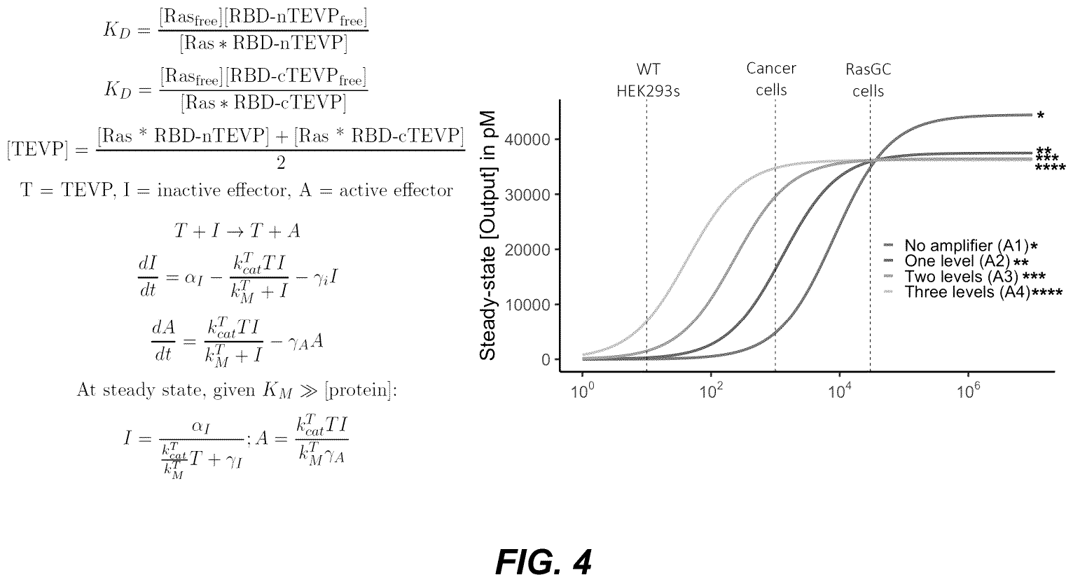

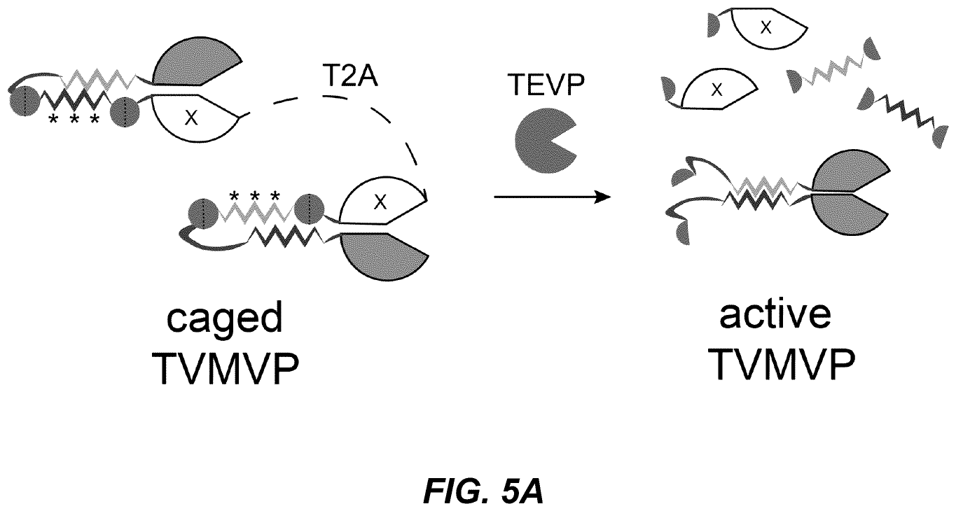

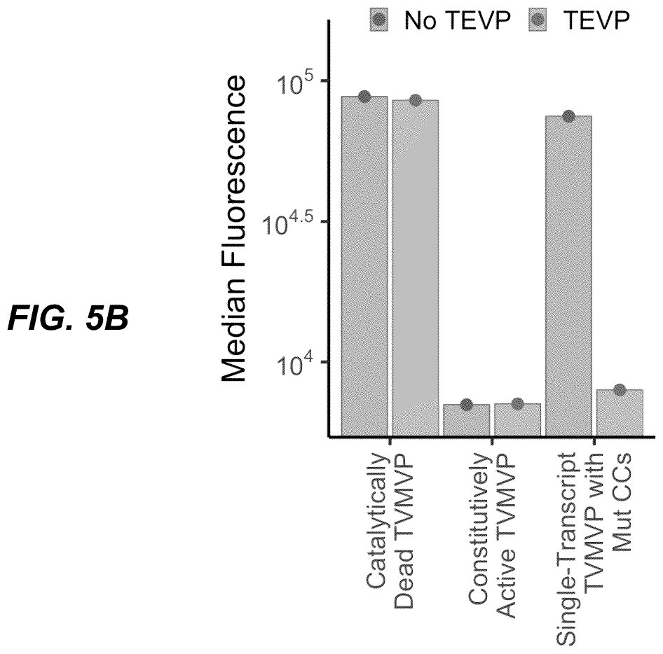

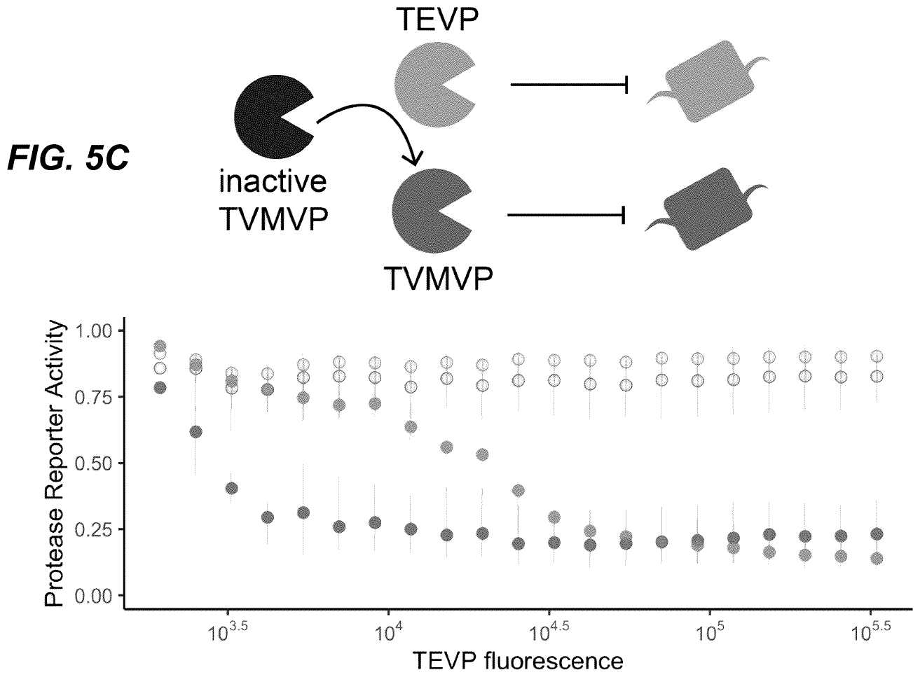

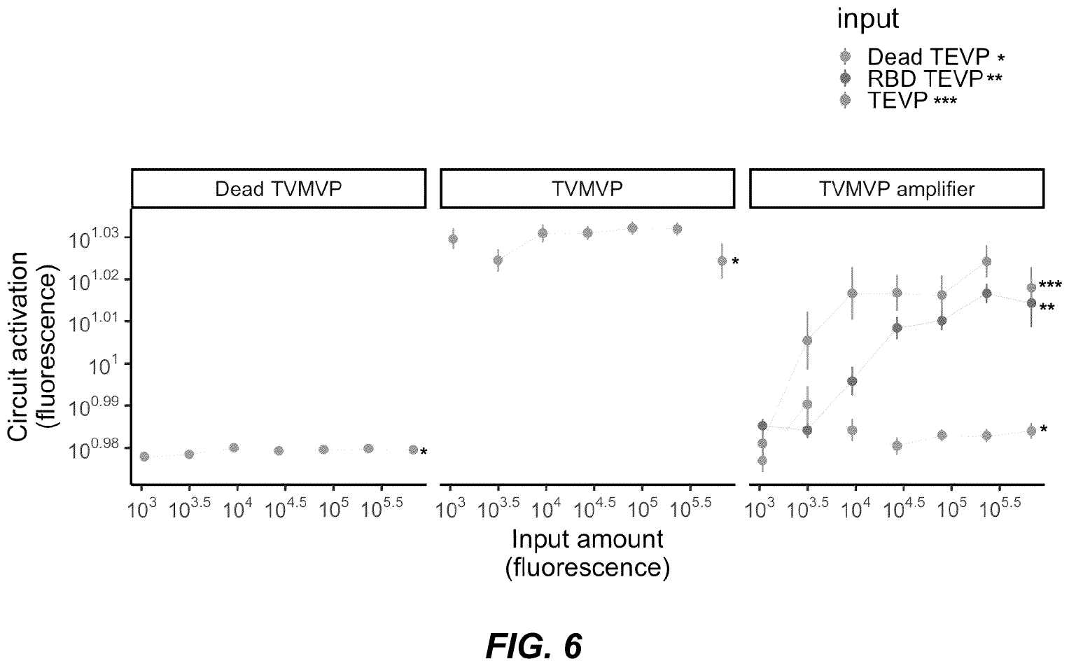

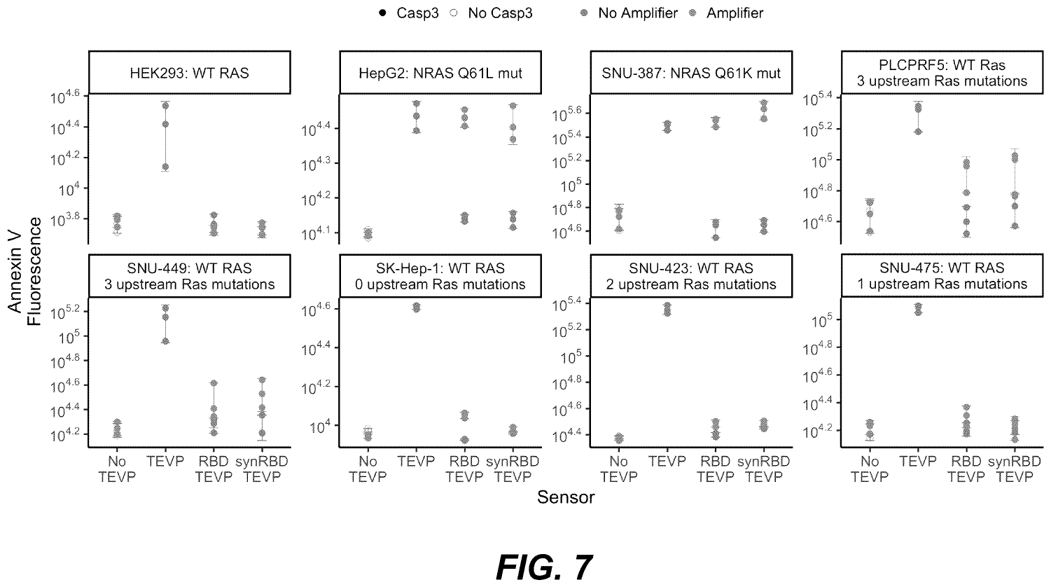

An ideal Ras cancer therapy should be able to specifically target cells with pathologically elevated Ras activity, independent of the underlying activation mechanism (e.g. specific mutations in upstream regulators of Ras, or different mutations in Ras itself). Previously, a protease-based circuit was developed that can detect hyperactivated Ras signaling (X. Gao et al, Programmable Protein Circuits in Living Cells, Science, 2018). In this system, elevated levels of Ras signaling lead to Ras protein nanoclusters on the inner cell membrane. When each half of a split TEV protease is fused to a ras binding domain (RBD), clustering reconstitutes TEV proteases, which can in turn trigger downstream responses. It was found that this RBD-mediated split-protease reconstitution enables sensing of Ras in artificially high Ras context. However, when delivered to cancer cell lines expressing hyperactive mutant ras variants at endogenous levels, the same circuit failed to activate, suggesting the need for signal amplification. Provided herein are therapeutic protein circuits that can conditionally kill cells with hyperactivated Ras signaling. Briefly, a Ras activity sensor was coupled with engineered signal processing and cell death executioner protein modules. A Ras activity sensor was engineered based on the CRAF Ras Binding Domain, an amplifier based on reconstitution of a split-caged protease, and a cell death executioner based on Casp3. FIG. 2 depicts a non-limiting exemplary schematic of a full circuit wherein (1) TEV protease-based Ras sensor detects Ras clustering on membrane, (2) TVMV protease-based module amplifies signal, and (3) Casp3 cell death executioner induces apoptosis. It was found that these modular components can be combined in a synthetic therapeutic circuit that can detect hyperactivated Ras activity and execute cell death. When transfected into cells, the resulting protein circuit kills cancer cell lines with elevated Ras activity. FIGS. 3A-3B depicts non-limiting exemplary data related to Ras binding domain (RBD) mediated split-protease reconstitution enabling sensing of Ras in an artificially high ras context but not endogenous levels. FIG. 4 depicts non-limiting exemplary data related to modeling identifying key parameters that control amplification properties. FIGS. 5A-5C depicts non-limiting exemplary schematics (FIG. 5A) and data (FIGS. 5B-5C) related to engineered proteases enabling a proteolysis-based amplification module. FIG. 6 depicts non-limiting exemplary data related to a protease amplifier increasing RBD-circuit sensitivity in a H358 NSCLC KRAS G12C mutant cell line. Further, the circuit can function across multiple mechanisms of Ras activation, and exhibits minimal effects in non-Ras hyperactive cells. FIG. 7 depicts non-limiting exemplary data related to a full circuit enabling sensitive and specific killing of mutant Ras cell lines while sparing wild type cells. Circuits provided herein can be encoded as mRNA and delivered transiently via lipid nanoparticles, ensuring rapid circuit expression and avoiding genome modification. The same type of amplification circuit can be employed to sense, amplify, and respond to signals from other oncogenic pathways, cell-type specific signaling cell states, or any user defined input, and to activate arbitrary responses. These results demonstrate how a rationally designed circuit can target Ras-hyperactivation, and provide a proof of principle for new circuit-level therapeutic strategies.

### Example 2

### Protein-Based Signal Amplification

To address the above-mentioned needs in the art, provided herein, in some embodiments, are protease-based amplifiers. These amplifiers can take a protease activity as an input, and control a distinct protease activity as an output. In some embodiments, this central module is a synthetic protease-activatable protease. In some embodiments the proteases are derived from plant viruses, making them orthogonal to mammalian proteases and enabling bioorthogonal operations.

A variety of protein-engineering strategies were designed and tested to create this module. First, an autoinhibitory domain (mutated, dominant negative canonical protease cleavage peptide) was attempted to be introduced to the TVMV protease, such that the peptide can occupy the active site but not be cleaved. However, this design had high background activity, as the autoinhibitory domain can transiently dissociate. In order to inhibit this dissociation, the TVMV protease was circularly permuted and the AI domain tied down on both sides to the protease. This construct was found to still have leaky activation. Moving to a different strategy, previous groups have found that using short linkers to circularly permute proteins can introduce structural strain and enzyme deactivation. Following cleavage of the short linkers, enzymes can then refold back to their original structure and restore activity. This strategy was applied to the TVMV protease and it was found that this strategy did not adequately strain the protease. Finally, blocking protease active site access with bulky synthetic proteins was also attempted to no avail.

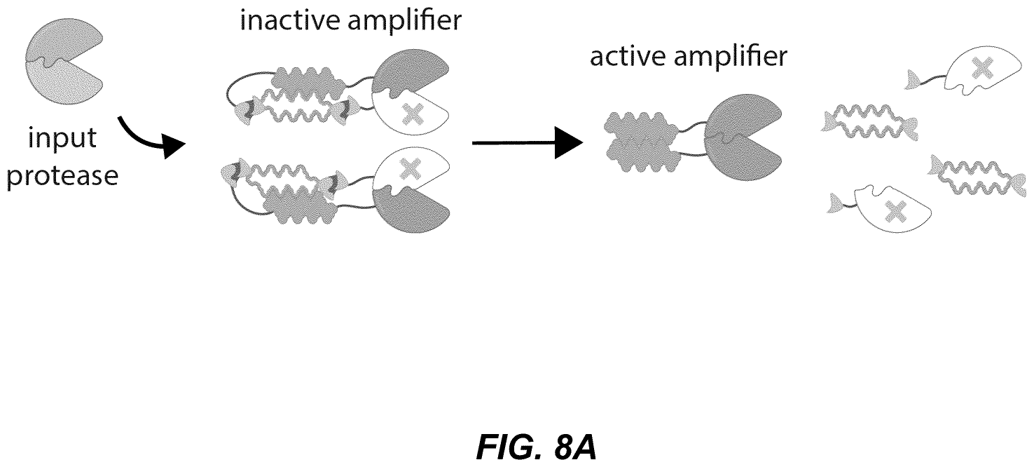

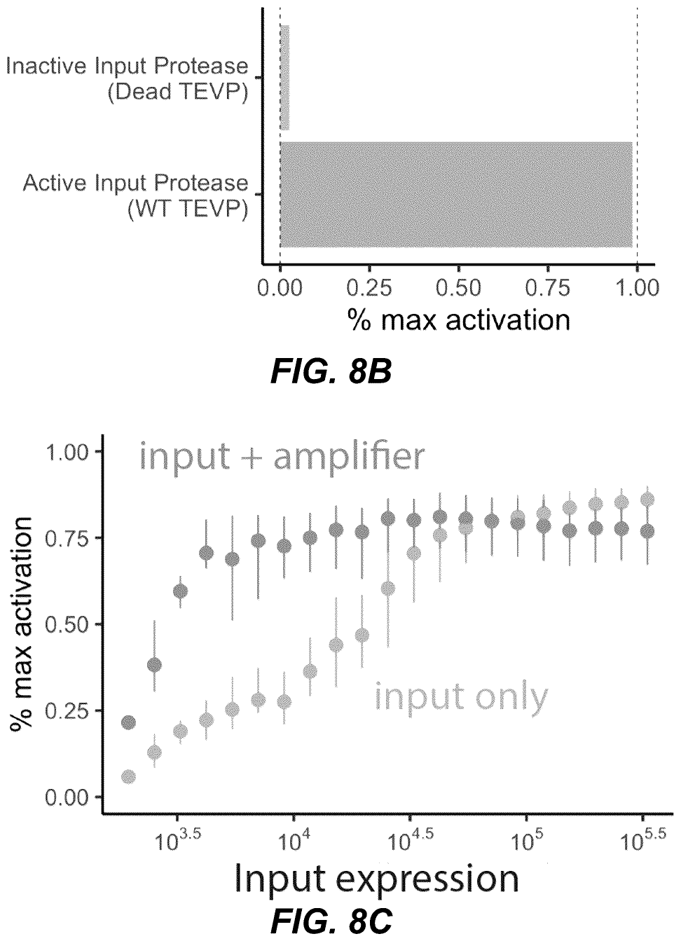

A strategy was then tested where each half of the split viral protease (TVMV) was caged with catalytically inactive complementary protease halves. First, only caging one half of the protease was attempted to but it was found to have high background activity. Next, both protease halves were caged. In its expressed form, both polypeptides are individually inactive and, due to caging, cannot reconstitute a functional output (TVMV) protease. However, proteolysis catalyzed by the input protease (TEVP) can cleave both caged protease halves between the functional domains and catalytically inactive caging domains. This releases the caging domains, allowing the functional domains to combine to form a functional TVMV protease (FIG. 8A). Complementary dimerization domains with varying affinities further facilitate this reconstitution in some embodiments. In some embodiments, high affinity dimerization domains (designed coiled coils) were attached to the functional protease halves, while attaching weaker dimerization domains to the inactive caging domains. Post cleavage, these properties can enable efficient displacement of the caging domains by the active domains. This strategy also works with de novo designed heterodimerization proteins in some embodiments.

The protease-activated protease was found to achieve signal amplification. In the absence of input activity, this “pro-TVMVP” has no measurable protease reporter activity. Upon induction of TEV protease, it became activated to levels comparable to full-length, active TVMVP (FIG. 8B). At the same level of low input TEVP activity, the pro-TVMVP activated at levels higher than TEVP alone, generating the desired signal amplification. (FIG. 8C). This double caging strategy can also work with other protease combinations (e.g. TVMVP-activated TEVP) and can be generalized to a diverse range of proteases with varying specificities to create multiple amplifiers that can operate independently in the same cell.

One application of the protease amplifiers provided herein is amplifying intrinsically weak signals from oncogenic signaling pathways. Previously, a protease-based circuit that can detect hyperactivated Ras signaling was developed. In this system, elevated levels of Ras signaling lead to Ras protein nanoclusters on the inner cell membrane. When each half of a split TEV protease is fused to a ras binding domain (RBD), clustering reconstitutes TEV proteases, which can in turn trigger downstream responses. It was found that this RBD-mediated split-protease reconstitution enables sensing of Ras in artificially high Ras context. However, when delivered to cancer cell lines expressing hyperactive mutant ras variants at endogenous levels, the same circuit failed to activate, suggesting the need for signal amplification.

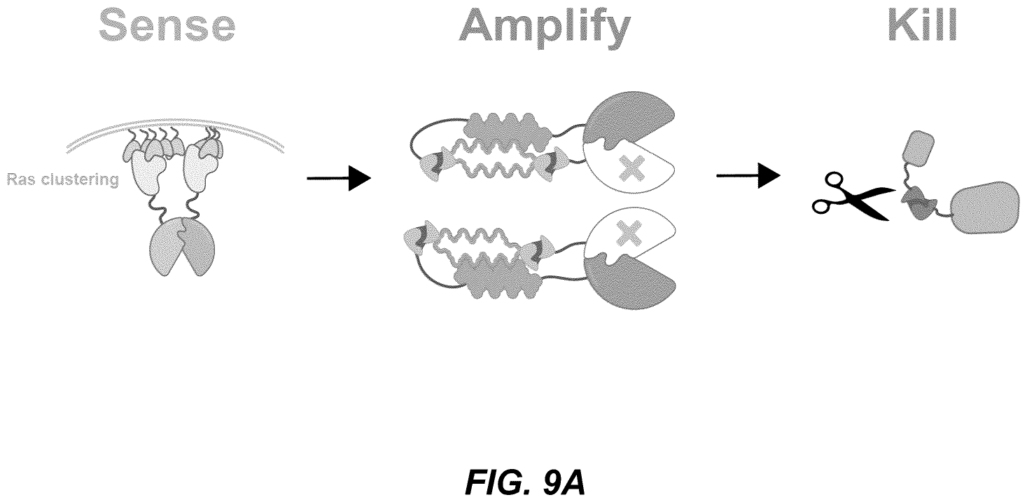

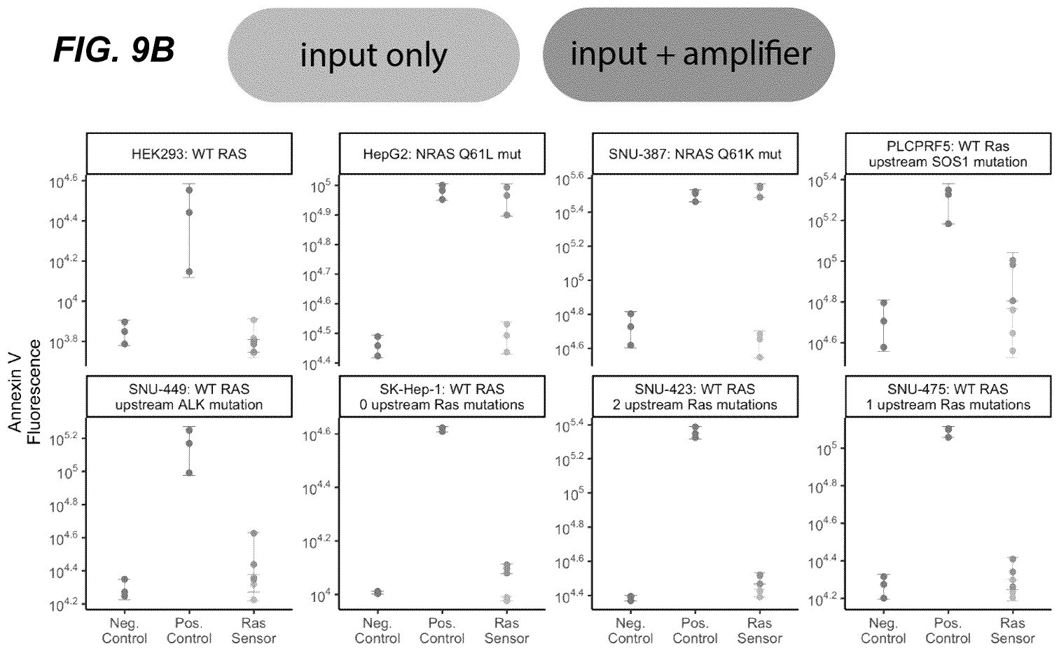

To amplify the intrinsically weak cancer Ras signal, the RBD-Ras activity sensor was coupled with the engineered TVMVP-based amplifier and, as output, a cell death executioner protein module (FIG. 9A). When transfected into cells, the resulting protein circuit selectively killed cancer cell lines with elevated Ras activity (FIG. 9B). Further, the circuit can function across multiple mechanisms of Ras activation, and exhibited minimal effects in non-Ras hyperactive cells.

The same type of amplification circuit can be generalized to sense, amplify, and respond to signals from other oncogenic pathways, cell-type specific signaling cell states, or any user defined input, and to activate arbitrary responses. For example, amplification circuits provided herein can be employed with sensors for a variety of cancer pathways such as Wnt/β-catenin, BCR-ABL, P53, and many others. Outside of oncogenic sensors, amplification circuits provided herein can be employed with sensors for fibrosis, senescence, and autoimmune diseases. The same amplifier provided herein can be added to any of those circuits if the initial signal also requires amplification. Additionally, there are provided, in some embodiments herein, response modules that allows user-defined control of cell death pathways such as apoptosis and pyroptosis. The disclosed synthetic protein circuits enable tunable, amplifying control of these response modules.

The same protease amplifier module can be used in diagnostic applications. Just as the ability to amplify and detect miniscule amounts of nucleic acids with PCR revolutionized molecular biology, so too could genetically encoded protein-level signal amplifiers enable a wide range of currently infeasible capture of endogenous levels of key molecules.

Together, these results demonstrate the invention of a protease-based signal amplification module and its application to a therapeutic protein circuit.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A synthetic protein circuit, comprising:
(i) an amplifier protein comprising a first part of a first protease domain, a first dimerization domain, a first cut site a protease in a protease active state is capable of cutting, a second dimerization domain, a second cut site a protease in a protease active state is capable of cutting, and a first caging domain;
(ii) a companion amplifier protein comprising a second part of a first protease domain, a third dimerization domain, a third cut site a protease in a protease active state is capable of cutting, a fourth dimerization domain, a fourth cut site a protease in a protease active state is capable of cutting, and a second caging domain;
(iii) one or more input protein(s), wherein said input protein(s) are capable of constituting a second protease in a second protease active state; and
(iv) one or more output protein(s), wherein said output protein(s) comprise a cut site the first protease in a first protease active state is capable of cutting, thereby modulating its expression, concentration, localization, stability, and/or activity.

2. The synthetic protein circuit of claim 1, wherein:
the first cut site and/or the second cut site is a cut site a second protease in a second protease active state is capable of cutting;
the third cut site and/or the fourth cut site is a cut site a second protease in a second protease active state is capable of cutting;
the first protease and/or the second protease comprises tobacco etch virus (TEV) protease, tobacco vein mottling virus (TVMV) protease, hepatitis C virus protease (HCVP), derivatives thereof, or any combination thereof.
said input protein(s) are configured to detect cell type and/or cell state;
said output protein(s) comprise one or more payload protein(s) and/or effector protein(s); and/or
said output protein(s) are capable of modulating cell type and/or cell state.

3. The synthetic protein circuit of claim 1, wherein:
the amplifier protein and the companion amplifier protein separately do not comprise a first protease capable of being in a first protease active state;
the first caging domain is a catalytically inactive version of the second part of a first protease domain; and/or
the second caging domain is a catalytically inactive version of the first part of a first protease domain.

4. The synthetic protein circuit of claim 1, wherein:
the first dimerization domain is capable of binding the second dimerization domain;
the first dimerization domain is capable of binding the third dimerization domain;
the third dimerization domain is capable of binding the fourth dimerization domain;
the affinity of the first dimerization domain for the second dimerization domain is weaker than the affinity of the first dimerization domain for the third dimerization domain;
and/or the affinity of the third dimerization domain for the fourth dimerization domain is weaker than the affinity of the third dimerization domain for the first dimerization domain.

5. The synthetic protein circuit of claim 1,
wherein intramolecular binding between the first dimerization domain and the second dimerization domain of the first amplifier protein is capable of preventing the first part of the first protease domain from associating with the second part of the first protease domain of the companion amplifier protein to form a first protease in a first protease active state; and/or
wherein intramolecular binding between the third dimerization domain and the fourth dimerization domain of the companion amplifier protein is capable of preventing the second part of the first protease domain from associating with the first part of a first protease domain of the amplifier protein to form a first protease in a first protease active state.

6. The synthetic protein circuit of claim 1, wherein a second protease in a second protease active state is capable of:
(i) cleaving the first cut site and/or second cut site of the amplifier protein, thereby forming a cleaved amplifier protein; and/or
(ii) cleaving the third cut site and/or fourth cut site of the companion amplifier protein, thereby forming a cleaved companion amplifier protein,
wherein a cleaved amplifier protein and a cleaved companion amplifier protein are capable of associating via intermolecular binding of the first dimerization domain and the third dimerization domain to form a first complex, wherein the first complex comprises a first protease capable of being in a first protease active state.

7. The synthetic protein circuit of claim 1, wherein one or more of the first dimerization domain, second dimerization domain, third dimerization domain, and fourth dimerization domain:
comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bc1-2 homology domain, a coiled-coil domain, a bZIP domain, portions thereof, a homo-dimerizing leucine zipper, a multimerizing leucine zipper, a hetero-dimerizing leucine zipper, a PDZ domain, a SH3 domain, aGBD domain, variants thereof, or any combination thereof; and/or
is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

8. The synthetic protein circuit of claim 1, wherein one or more of the first dimerization domain, second dimerization domain, third dimerization domain, and fourth dimerization domain.

9. The synthetic protein circuit of claim 1, wherein the amplifier protein and companion amplifier protein are configured to form an amplification module, and wherein:
(i) the number of molecules of the first protease in a first protease active state is at least 1.1-fold greater than the number of molecules of the second protease in a second protease active state; and/or
(ii) the rate of first protease-mediated cleavage is at least 1.1-fold greater than the rate of second protease-mediated cleavage,
thereby achieving signal amplification.

10. The synthetic protein circuit of claim 9, wherein configuring the amplifier protein and companion amplifier protein to form an amplification module comprises one or more of:
introducing one or more amino acid substitutions into the cut site(s) to increase cleavage efficiency;
introducing one or more amino acid substitutions into the first dimerization domain and/or the third dimerization domain to increase affinity for each other;
introducing one or more amino acid substitutions into the first dimerization domain and/or the second dimerization domain to decrease affinity for each other;
introducing one or more amino acid substitutions into the third dimerization domain and/or the fourth dimerization domain to decrease affinity for each other;
introducing one or more amino acid substitutions into the first part of a first protease domain and/or the second part of a first protease domain to increase catalytic activity; and/or
increasing the relative levels of the amplifier protein and the companion amplifier protein.

11. The synthetic protein circuit of claim 9, wherein:
the presence of the amplification module decreases the level of input signal required for a synthetic protein circuit to generate a given level of output by at least about 1.1-fold as compared to a synthetic protein circuit which does not comprise the amplification module;
and/or the presence of the attenuation module increases the level of input signal required for a synthetic protein circuit to generate a given level of output by at least about 1.1-fold as compared to a synthetic protein circuit which does not comprise the attenuation module.

12. The synthetic protein circuit of claim 1, wherein the amplifier protein and companion amplifier protein are configured to form an attenuation module, wherein:
(i) the number of molecules of the first protease in a first protease active state is at least 1.1-fold less than the number of molecules of the second protease in a second protease active state; and/or
(ii) the rate of first protease-mediated cleavage is at least 1.1-fold less than the rate of second protease-mediated cleavage,
thereby achieving signal attenuation.

13. The synthetic protein circuit of claim 11, wherein configuring the amplifier protein and companion amplifier protein to form an attenuation module comprises one or more of:
introducing one or more amino acid substitutions into the cut site(s) to decrease cleavage efficiency;
introducing one or more amino acid substitutions into the first dimerization domain and/or the third dimerization domain to decrease affinity for each other;
introducing one or more amino acid substitutions into the first dimerization domain and/or the second dimerization domain to increase affinity for each other;
introducing one or more amino acid substitutions into the third dimerization domain and/or the fourth dimerization domain to increase affinity for each other;
introducing one or more amino acid substitutions into the first part of a first protease domain and/or the second part of a first protease domain to decrease catalytic activity; and/or
reducing the relative levels of the amplifier protein and the companion amplifier protein.

14. The synthetic protein circuit of claim 11, wherein:
the presence of the amplification module increases the level of output generated by a given level of input signal by at least about 1.1-fold as compared to a synthetic protein circuit which does not comprise the amplification module; and/or
the presence of the attenuation module decreases the level of output generated by a given level of input signal by at least about 1.1-fold as compared to a synthetic protein circuit which does not comprise the attenuation module.

15. The synthetic protein circuit of claim 1, wherein the input protein(s) comprise:
a first input protein comprising a first signal transducer binding domain and a first part of a second protease domain, wherein the first signal transducer binding domain is capable of binding a first signal transducer to form a first signal transducer-bound input protein; and
a second input protein comprising a second signal transducer binding domain and a second part of the second protease domain, wherein the second signal transducer binding domain is capable of binding a second signal transducer to form a second signal transducer-bound input protein,
wherein the first part of the second protease domain and the second part of the second protease domain have weak association affinity, and wherein the first part of the second protease domain and the second part of the second protease domain are capable of associating with each other to constitute a second protease capable of being in a second protease active state when the first signal transducer and the second signal transducer are in close proximity at an association location.

16. The synthetic protein circuit of claim 15, wherein:
the first signal transducer binding domain of the first input protein and the second signal transducer binding domain of the second input protein are identical;
the first transducer and the second transducer are identical;
the first signal transducer, the second signal transducer, or both, are capable of being localized at the association location;
the association location comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, filopodium, ruffle, lamellipodium, sarcomere, focal contact, podosome, ribosome, microsome, plasma membrane, nuclear membrane, chloroplast, cell wall, or any combination thereof;
the first signal transducer when in a first signal transducer active state, the second signal transducer when in a second signal transducer active state, or both, are capable of being localized at the association location;
the first signal transducer when in a first inactive state, the second signal transducer when in a second inactive state, or both, are capable of being localized at the association location;
the first part of the second protease domain and the second part of the second protease domain have the weak association affinity when the first signal transducer is in a first signal transducer inactive state and/or the second signal transducer inactive state; and/or
the first part of the second protease domain and the second part of the second protease domain are incapable of associating to form the second protease in the second protease active state when the first signal transducer is in a first signal transducer inactive state and/or the second signal transducer is in a second signal transducer inactive state.

17. The synthetic protein circuit of claim 15, wherein the effector protein comprises a cut site the first protease in the first protease active state is capable of cutting, and wherein:
the effector protein is changed into a effector destabilized state, a effector delocalized state, and/or a effector inactivate state after the first protease in the first protease active state cuts the cut site of the effector protein;
the effector protein comprises a degron, wherein the first protease in the first protease active state is capable of cutting the cut site of the effector protein to expose the degron, and wherein the degron of the effector protein being exposed changes the effector protein to an effector destabilized state;
the effector protein is changed into a effector stabilized state, a effector localized state, and/or a effector activate state after the first protease in the first protease active state cuts the cut site of the effector protein;
the effector protein comprises a degron, wherein the first protease in the first protease active state is capable of cutting the cut site of the effector protein to hide the degron, and wherein the degron of the effector protein being hidden changes the effector protein to an effector stabilized state;
the effector protein comprises Caspase-3, Caspase 7, Caspase-9, Caspase-8, Bax, Bid, Bad, Bak, BCL2L11, p53, PUMA, Diablo/SMAC, S-TRAIL, or any combination thereof;
the first signal transducer binding domain and/or the second signal transducer binding domain comprises a RAS binding domain (RBD) and/or RAS association domain (RAD);
the first signal transducer, the second signal transducer, or both are endogenous proteins; and/or
the first signal transducer, the second signal transducer, or both comprise AKT, PI3K, MAPK, p44/42 MAP kinase, TYK2, p38 MAP kinase, PKC, PKA, SAPK, ELK, JNK, cJun, RAS, KRAS, NRHAS, HRAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCγ, NF-kB, FAK, CREB, αIIIβ3, FcεRI, BAD, p70S6K, STAT1, STAT2, STATS, STATS, STAT6, or any combination thereof.

18. A nucleic acid composition, comprising:
one or more polynucleotides encoding the synthetic protein circuit of claim 1, wherein the one or more polynucleotides comprise:
one or more first polynucleotides encoding an amplifier protein,
one or more second polynucleotides encoding a companion amplifier protein,
one or more third polynucleotides encoding one or more input protein(s), and/or
one or more fourth polynucleotides encoding one or more output protein(s).

19. An engineered cell or a population of engineered cells, comprising: the synthetic protein circuit of claim 1.

20. A method of treating or preventing a disease or disorder in a subject in need thereof, comprising:
expressing the synthetic protein circuit of claim 1 in a cell of a subject in need thereof.
